(19) United States
(12) Patent Application Publication
(10) Pub. No.: US 2025/0050116 A1
(43) Pub. Date: Feb. 13, 2025

(54) Automated Selection of Electrodes and Stimulation Parameters in a Deep Brain Stimulation System

(71) Applicant: Boston Scientific Neuromodulation Corporation, Valencia, CA (US)

(72) Inventors: Lisa Moore, Glendale, CA (US); Bhaskar Soni, Gwalior (IN); Rachel Fischell, West Hills, CA (US); Chirag Shah, Valencia, CA (US)

(21) Appl. No.: 18/797,077

(22) Filed: Aug. 7, 2024

Related U.S. Application Data
(60) Provisional application No. 63/518,549, filed on Aug. 9, 2023.

(30) Foreign Application Priority Data
Aug. 8, 2023 (IN) 202311053279

(51) Int. Cl. A61N 1/372; A61N 1/05; A61N 1/36; G16H 20/40

(52) U.S. Cl. CPC A61N 1/37241; A61N 1/0534; A61N 1/36157; A61N 1/36171; A61N 1/36175; A61N 1/36185; A61N 1/37247; A61N 1/37264; G16H 20/40

(57) ABSTRACT

A programming algorithm is disclosed to efficiently select stimulation parameters for a patient having a Deep Brain Stimulation (DBS) implant. The algorithm preferably first determines an optimal longitudinal position (Lopt) and amplitude (Iopt1) for stimulation along the lead. This occurs by the algorithm efficiently suggested various values for L and I at which stimulation can be tried on the patient and scored. Once Lopt is determined, the algorithm can also determine an optimal rotational angle (θopt) and amplitude (Iopt2) for stimulation around the lead at the optimized longitudinal position Lopt. This also occurs by the algorithm efficiently suggesting various values for θ and I at which stimulation can be tried on the patient and scored. Despite suggesting next L,I or θ,I values to test, the algorithm allows flexibility to allow a user to test any desired stimulation parameters beyond those suggested.

### CROSS REFERENCE TO RELATED APPLICATIONS

This application claims priority to U.S. Provisional Patent Application Ser. No. 63/518,549, filed Aug. 9, 2023, and to Indian Provisional Patent Application Ser. No. 20/231,1053279, filed Aug. 8, 2023. These priority applications are incorporated herein by reference in their entireties.

### FIELD OF THE INVENTION

This application relates to Implantable Stimulator Devices (ISD), and more specifically to an algorithm for selecting electrodes and stimulation parameters in an ISD such as a Deep Brain Stimulation (DBS) device.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) system, such as that disclosed in U.S. Patent Application Publication 2020/0001091, which is incorporated herein by reference. However, the present invention may find applicability with any implantable neurostimulator device system, including Spinal Cord Stimulation (SCS) systems, Vagus Nerve Stimulation (VNS) system, Sacral Nerve Stimulation (SNS) systems, Peripheral Nerve Stimulation (PNS) systems, and the like.

A DBS system typically includes an Implantable Pulse Generator (IPG) 10 shown in FIG. 1A. The IPG 10 includes a biocompatible device case 12 that holds the circuitry and a battery 14 for providing power for the IPG to function, although the IPG 10 can also lack a battery and can be wirelessly powered by an external source. The IPG 10 is coupled to tissue-stimulating electrodes 16 via one or more electrode leads 18 or 19, which are shown in more details in FIGS. 1B and 1C.

FIG. 1B shows a lead 18 having eight ring-shaped electrodes 16 which are circumferential around the lead and which are located at different longitudinal positions along a central axis 15. Lead 18 is referred to herein as a “non-directional lead,” because the ring-shaped electrodes span 360 degrees around the long axis 15 of the lead 18, and thus cannot direct stimulation to different rotational positions around the axis 15.

FIG. 1C shows a lead 19 having sixteen electrodes, but not all of the electrodes are ring-shaped. Electrodes E1-E4 at a proximal end of the lead 19 are ring-shaped. Electrodes E5-E16, by contrast, comprise split-ring electrodes. Certain of these split ring electrodes (e.g., E5-E7) are located at the same longitudinal position along the axis 15, but each spans less than 360 degrees around the axis. For example, each of electrodes E5-E7 may span 90 degrees around the axis 15, with each being separated from the others by gaps of 30 degrees. Electrodes E8-E10 also comprise split-ring electrodes, but are located at a different longitudinal position. Lead 19 is referred to herein as a “directional lead,” because at least some of the electrodes at a given longitudinal position (e.g., E5, E6, E7) span less than 360 degrees, meaning that those electrodes can direct stimulation to different rotational positions (and hence different brain tissues) around the axis 15. In other designs of a directional lead 19, all electrodes can be split-ring, or there could be different numbers of split-ring electrodes at each longitudinal position (i.e., more or less than three).

Lead wires 20 within the leads are coupled to the electrodes 16 and to proximal contacts 21 insertable into lead connectors 22 fixed in a header 23 on the IPG 10, which header can comprise an epoxy for example. Alternatively, the proximal contacts 21 may connect to lead extensions (not shown) which are in turn inserted into the lead connectors 22. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are four percutaneous leads 18 or 19, and thus the header 23 may include a 2×2 array of eight-electrode lead connectors 22. However, the type and number of leads, and the number of electrodes on each, in an IPG is application specific and therefore can vary. The conductive case 12 can also comprise an electrode (Ec).

In a DBS application, as is useful in the treatment of tremor in Parkinson's disease for example, the IPG 10 is typically implanted under the patient's clavicle (collarbone). Leads 18 or 19 (perhaps as extended by lead extensions, not shown) are tunneled through and under the neck and the scalp, with the electrodes 16 implanted through holes drilled in the skull and positioned for example in the subthalamic nucleus (STN) and the pedunculopontine nucleus (PPN) in each brain hemisphere. The IPG 10 can also be implanted underneath the scalp closer to the location of the electrodes' implantation, as disclosed for example in U.S. Pat. No. 10,576,292. The IPG lead(s) 18 or 19 can be integrated with and permanently connected to the IPG 10 in other solutions.

IPG 10 can include an antenna 27a allowing it to communicate bi-directionally with a number of external devices and systems discussed subsequently. Antenna 27a as shown comprises a conductive coil within the case 12, although the coil antenna 27a can also appear in the header 23. When antenna 27a is configured as a coil, communication with external systems preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like. Although not shown, the IPG 10 may include an additional coil to receive wireless power from an external source, or to charge the IPG's battery 14.

Stimulation in IPG 10 is typically provided by pulses each of which may include a number of phases such as 30a and 30b, as shown in the example of FIGS. 2A and 2B. In the example shown, such stimulation is monopolar, meaning that a current is provided between at least one selected lead-based electrode (e.g., E1) and the case electrode Ec 12. Stimulation could be bipolar, in which a current is provided between at least two lead-based electrodes. Stimulation parameters typically include amplitude (current I, although a voltage amplitude V can also be used); frequency (F); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue Z. The case electrode Ec has been selected as an anode (again during first phase 30a), and thus provides pulses which source a corresponding positive current of amplitude +I to the tissue Z. Note that at any time the total current sunk from the tissue (e.g., −I at E1 during phase 30a) equals the total current sourced to the tissue (e.g., +I at Ec during phase 30a). The polarity of the currents at these electrodes can be changed: for example, during first phase 30a, Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40ᵢ and one or more current sinks 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40ᵢ and NDACs 42; allows any of the electrodes 16 and the case electrode Ec 12 to act as anodes or cathodes to create a current (such as the pulses described earlier) through a patient's tissue, Z, hopefully with good therapeutic effect. In the example shown, and consistent with the first pulse phase 30a of FIG. 2A, electrode E1 has been selected as a cathode electrode to sink current from the tissue Z and case electrode Ec has been selected as an anode electrode to source current to the tissue Z. Thus PDAC 40c and NDAC 421 are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40ᵢ and the electrode nodes ei 39, and between the one or more NDACs 42; and the electrode nodes. Switching matrices allow one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs) within the IPG 10, as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38, as is well known. FIG. 3 also shows that stimulation circuitry 28 can include passive recovery switches 41ᵢ, which are described further in U.S. Patent Application Publications 2018/0071527 and 2018/0140831. Passive recovery switches 41ᵢ may be closed to passively recover any charge remaining on the DC-blocking capacitors Ci 38 after issuance of the last pulse phase (e.g., second pulse phase 30b) to recover charge without actively driving a current using the DAC circuitry, as shown during duration 30c. Again, passive charge recovery is well known and not further described. Although not shown, the stimulation pulses may also be monophasic comprising single actively-driven phases (e.g., 30a), and followed by passive charge recovery (e.g., 30c).

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information and measurements, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60′s user interface enables a patient to adjust stimulation parameters, although it may have limited functionality when compared to systems 70 and 80, described shortly. The external controller 60 can have one or more antennas capable of communicating with a compatible antenna in the IPG 10 (27a or 27b), such as a near-field magnetic-induction coil antenna 64a and/or a far-field RF antenna 64b.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller. A communication “wand” 76 coupleable to suitable ports on the computing device can include an IPG-compliant antenna such as a coil antenna 74a or an RF antenna 74b. The computing device itself may also include one or more RF antennas 74b. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with IPG communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

### SUMMARY

A system is disclosed, which may comprise: a non-transitory computer-readable medium comprising an algorithm configured to be executed by control circuitry of an external system configured to program an implantable stimulator device (ISD) of a patient, wherein the ISD comprises a lead with a plurality of electrodes for providing stimulation in accordance with a plurality of stimulation parameters.

The algorithm may be configured to: allow a user to define or accept one or more rules for the stimulation parameters, wherein the one or more rules are consistent with a parameter space to be tested; iteratively test sets of stimulation parameters, wherein at each iteration the algorithm is configured to present a set of stimulation parameters to a user within the parameter space, receive either (i) an indication that the user accepts the set of stimulation parameters, or (ii) user-defined stimulation parameters, assess compliance of the user-defined stimulation parameters, if received, with the one or more rules, program the ISD to provide stimulation using (i) the set of stimulation parameters if accepted, else (ii) the user-defined stimulation parameters, record an entry comprising the stimulation and at least one score indicative of the efficacy of the simulation, flag the entry with a first flag if the stimulation was provided using the user-defined stimulation parameters and if the user-defined stimulation parameters are not compliant with the one or more rules, and determine a next set of stimulation parameters to test within the parameter space using entries that are not flagged with the first flag; and determine as optimal stimulation for the patient the stimulation associated with the entry having a best score.

In one example, the score is indicative of the efficacy of the stimulation in treating a symptom of the patient and/or a side effect caused by the stimulation. In one example, at least one of the one or more rules comprises a rule requiring the stimulation to have a pulse width or frequency that is constant. In one example, at least one of the one or more rules comprises a rule requiring the stimulation to have a consistent polarity. In one example, the parameter space comprises a range of longitudinal positions on the lead. In one example, at least one of the one or more rules comprises a rule requiring the stimulation to have a longitudinal position within the range of longitudinal positions. In one example, at least one of the one or more rules comprises a rule requiring the stimulation to be symmetric around the lead. In one example, the parameter space further comprises a range of amplitudes. In one example, at least one of the one or more rules comprises a rule requiring the stimulation to have an amplitude with the range of amplitudes. In one example, determining the next set of stimulation parameters to test comprises determining a longitudinal position along the lead and an amplitude. In one example, the parameter space comprises a range of rotational positions around the lead. In one example, at least one of the one or more rules comprises a rule requiring the stimulation to have a rotational position within the range of rotational positions. In one example, the parameter space further comprises a range of amplitudes. In one example, at least one of the one or more rules comprises a rule requiring the stimulation to have an amplitude with the range of amplitudes. In one example, at least one of the one or more rules comprises a rule requiring the stimulation to be at a longitudinal position along the lead, or within a range of longitudinal position along the lead. In one example, determining the next set of stimulation parameters to test comprises determining a rotational position around the lead and an amplitude. In one example, during iteratively testing the sets of stimulation parameters, the algorithm is further configured to selectively determine if one or more stopping criterium have been met, and thus stop iteratively testing the sets of stimulation parameters, before determining the optimal stimulation for the patient. In one example, if the stimulation was provided using the user-defined stimulation parameters, the algorithm is configured to not determine if the one or more stopping criterium have been met. In one example, the system further comprises the ISD. In one example, the system further comprises the external system.

A method is disclosed that is performed on an external system configured to program an implantable stimulator device (ISD) of a patient, wherein the ISD comprises a lead with a plurality of electrodes for providing stimulation in accordance with a plurality of stimulation parameters. The method may comprise: allowing a user to define or accept at the external system one or more rules for the stimulation parameters, wherein the one or more rules are consistent with a parameter space to be tested; iteratively testing sets of stimulation parameters, wherein at each iteration the method presents at the external system a set of stimulation parameters to a user within the parameter space, receives either (i) an indication that the user accepts the set of stimulation parameters, or (ii) user-defined stimulation parameters, assesses compliance of the user-defined stimulation parameters, if received, with the one or more rules, programs the ISD to provide stimulation using (i) the set of stimulation parameters if accepted, else (ii) the user-defined stimulation parameters, and records an entry comprising the stimulation and at least one score indicative of the efficacy of the simulation, flags the entry with a first flag if the stimulation was provided using the user-defined stimulation parameters and if the user-defined stimulation parameters are not compliant with the one or more rules, and determines a next set of stimulation parameters to test within the parameter space using entries that are not flagged with the first flag; and determining, at the external system, as optimal stimulation for the patient the stimulation associated with the entry having a best score.

### BRIEF DESCRIPTION OF THE DRAWINGS

FIG. 1A shows an Implantable Pulse Generator (IPG), in accordance with the prior art. FIG. 1B shows a percutaneous lead having ring electrodes, and FIG. 1C shows a percutaneous lead having split ring electrodes, in accordance with the prior art.

FIGS. 2A show an example of stimulation pulses (waveforms) producible by the IPG, and FIG. 2B shows the flow of current in the tissue, in accordance with the prior art.

FIG. 3 shows an example of stimulation circuitry useable in the IPG, in accordance with the prior art.

FIG. 4 shows various external systems capable of communicating with and programming stimulation in an IPG, in accordance with the prior art.

FIG. 5A shows a Graphical User Interface (GUI) operable on an external system such as a clinician programmer, which is capable of programming a stimulation program for the IPG. FIG. 5B shows waveforms produced at the electrodes through use of the GUI of FIG. 5A.

FIG. 6 shows a programming algorithm for optimizing stimulation in a patient, including the ability to determine an optimal longitudinal position and rotational angle of the stimulation, as well as an optimal amplitude.

FIG. 7 shows an example of a GUI representation of a lead for which stimulation is optimized, and further shows parameters spaces useful to understanding the optimization algorithm.

FIG. 8 shows steps involving how an optimal longitudinal position (Lopt) and an optimal amplitude (Iopt1) are determined in a first part of the algorithm.

FIG. 9 shows how various potential (L I) values can be excluded.

FIG. 10 shows sub-steps in the algorithm relevant to selecting a next (L,I) value to be tested.

FIGS. 11A-11F show further details of the sub-steps shown in FIG. 10, including the selection of a next (L,I) point to test, and considerations of how the algorithm can assess tissue imaging information during longitudinal optimization.

FIG. 12 shows steps involving how an optimal rotational angle (θopt) and an optimal amplitude (Iopt2) are determined at Lopt in a second part of the algorithm.

FIG. 13 shows further details involving the selection of a next (θ,I) point to test.

FIG. 14 shows the GUI that may be used to implement the optimization algorithm, and to display information that may be useful to the clinician as the algorithm operates.

FIG. 15 shows a modification to the algorithm in which the clinician can enter a plurality of scores for each tested stimulation parameter set at each iteration of the algorithm.

FIG. 16 shows a modified version of the algorithm which provides a user flexibility to select stimulation parameters to test that are not necessarily suggested by the algorithm.

FIG. 17 shows modified steps involving how an optimal longitudinal position (Lopt) and an optimal amplitude (Iopt1) are determined in a first part of the modified algorithm.

FIGS. 18A-18C show examples of the resulting data sets from operation of the first part of the algorithm of FIG. 17.

FIG. 19 shows modified steps involving how an optimal rotational position (θopt) and an optimal amplitude (Iopt2) are determined in a second part of the modified algorithm.

FIG. 20 shows an example of the resulting data set from operation of the second part of the algorithm of FIG. 19.

### DETAILED DESCRIPTION

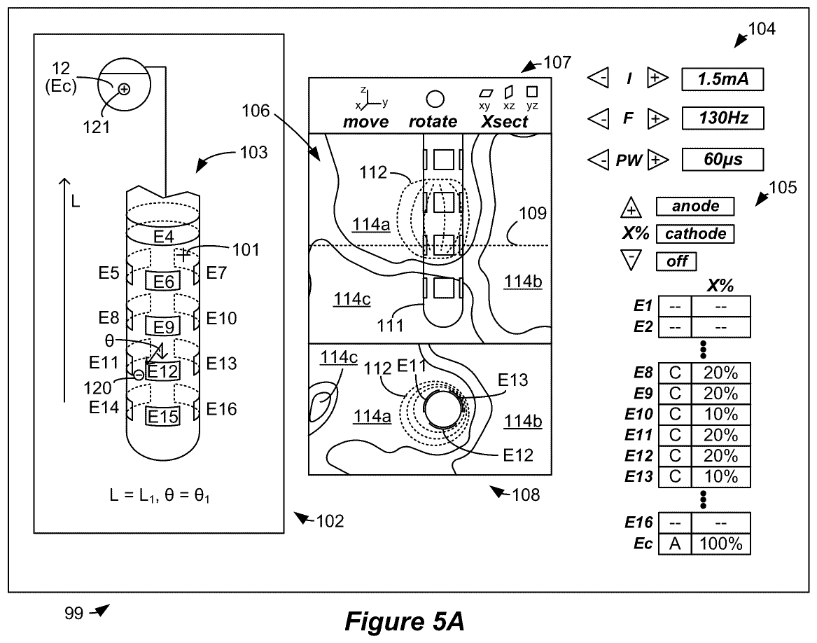

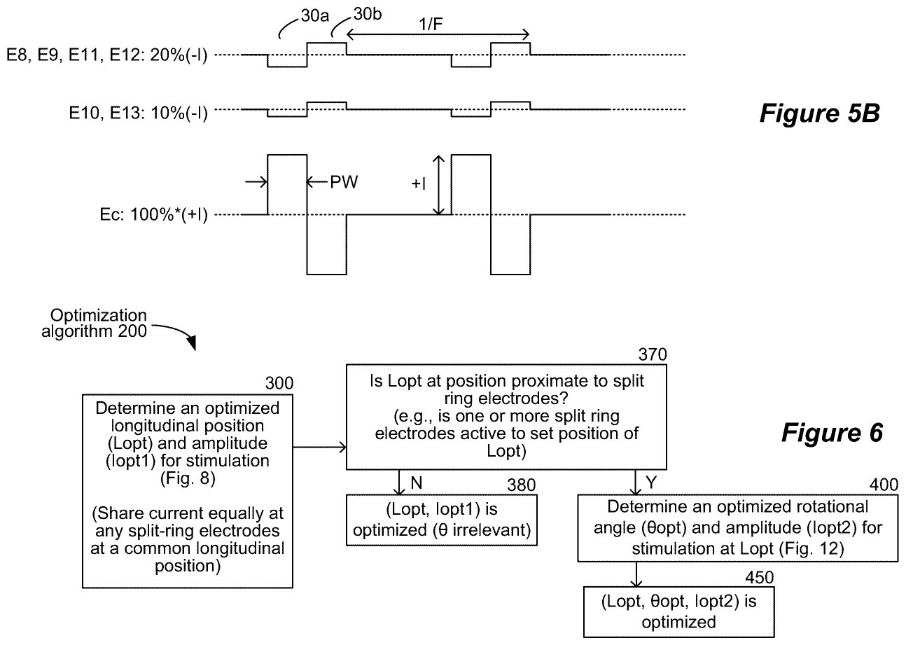

FIG. 5A shows an example of GUI 99 renderable by the external system software 96 on the display of an external system, such as the clinician programmer 70 mentioned earlier. GUI 99 is particularly useful in a DBS context because it provides a clinician with a visual indication of how stimulation selected for a patient will interact with the brain tissue in which the electrodes are implanted. GUI 99 can be used during surgical implantation of the leads 18 or 19 and its IPG 10, but can also be used after implantation to assist in selecting or adjusting a therapeutically useful stimulation program for the patient. The GUI 99 can be controlled by a cursor 101 that the user can move using a mouse connected to the clinician programmer 70 for example.

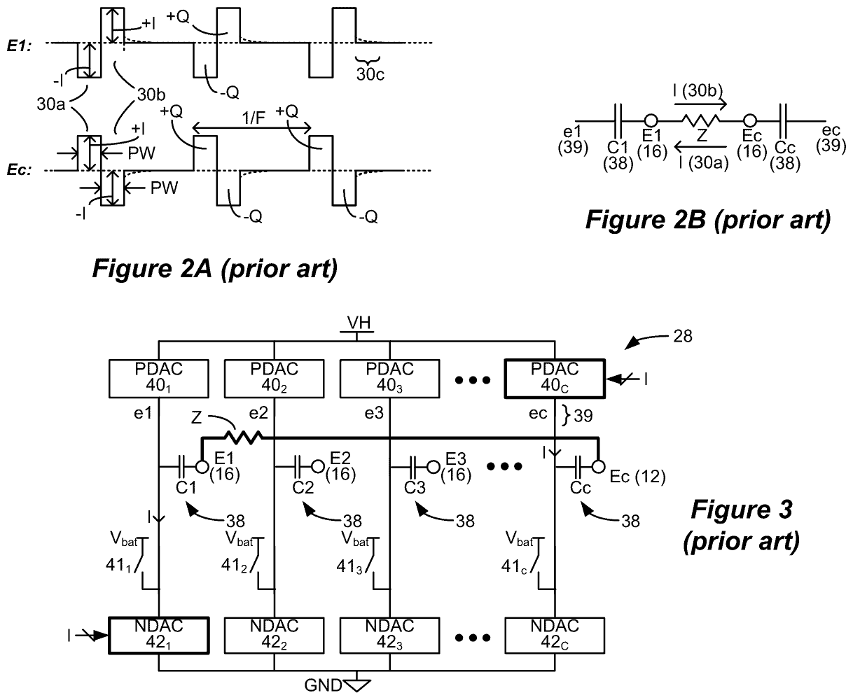

The GUI 99 may include a waveform interface 104 where various aspects of the stimulation can be selected or adjusted. For example, waveform interface 104 allows a user to select an amplitude (e.g., a current I), a frequency (F), and a pulse width (PW) of the stimulation pulses. Waveform interface 104 can be significantly more complicated, particularly if the IPG 10 supports the provision of stimulation that is more complicated than a repeating sequence of pulses. Waveform interface 104 may also include inputs to allow a user to select whether stimulation will be provided using biphasic (FIG. 2A) or monophasic pulses, or in bursts of pulses, and to select whether passive charge recovery will be used, although again these details aren't shown for simplicity.

The GUI 99 may also include an electrode configuration interface 105 which allows the user to select a particular electrode configuration specifying which electrodes should be active to provide the stimulation, and with which polarities and relative magnitudes. In this example, the electrode configuration interface 105 allows the user to select whether an electrode should comprise an anode (A) or cathode (C) or be off, and allows the amount of the total anodic or cathodic current +I or −I (specified in the waveform interface 104) that each selected electrode will receive to be specified in terms of a percentage, X. For example, in FIG. 5A, the case electrode 12 Ec is specified to be an anode that receives X=100% of the current I as an anodic current +I (e.g., during first pulse phase 30a if biphasic pulses are used; see FIG. 2A). The corresponding cathodic current −I is split between cathodes electrodes E8, E9, E11, and E12 (20% or 0.2*−I each) and E10 and E13 (10% or 0.1* −I each) (again during first pulse phase 30a). The waveforms resulting at the electrodes from this electrode configuration are shown in FIG. 5B. Note that two or more electrodes can be chosen to act as anodes or cathodes at a given time, allowing the electric field in the tissue to be shaped, as explained further below.

Once the waveform parameters (104) and electrode configuration parameters (105) are determined, they can be sent from the clinician programmer 70 to the IPG 10, so that the IPG's stimulation circuitry 28 (FIG. 3) can be programmed (the various NDACs and PDACs) to produce the desired currents at the selected electrodes with the proper timing. For example, PDAC 40_{C} at case electrode Ec) would be programmed to produce 100%*+I, and NDAC 42₁₂ (at electrode E12) would be programmed to produce 20% of −I, etc. Together, the various waveform parameters and electrode configuration parameters comprise stimulation parameters, which together comprise a stimulation program.

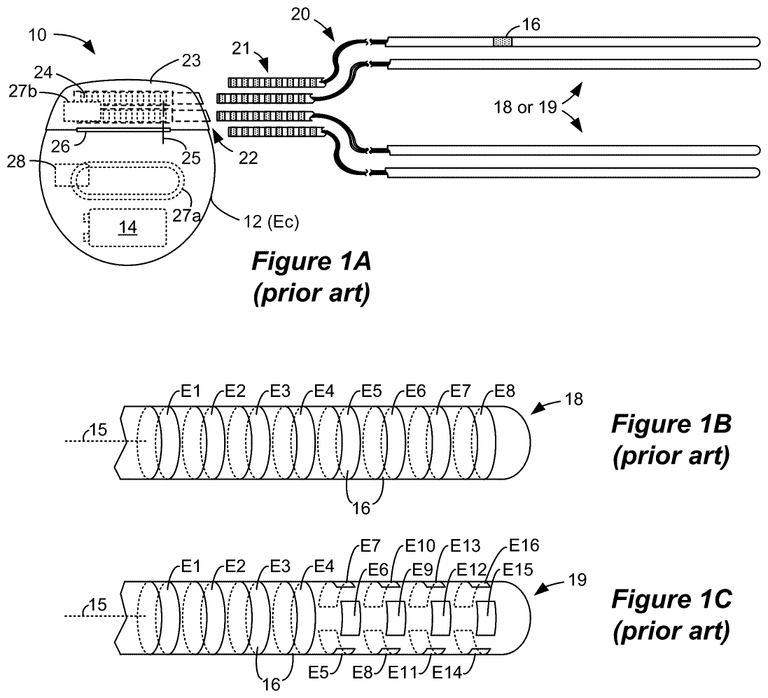

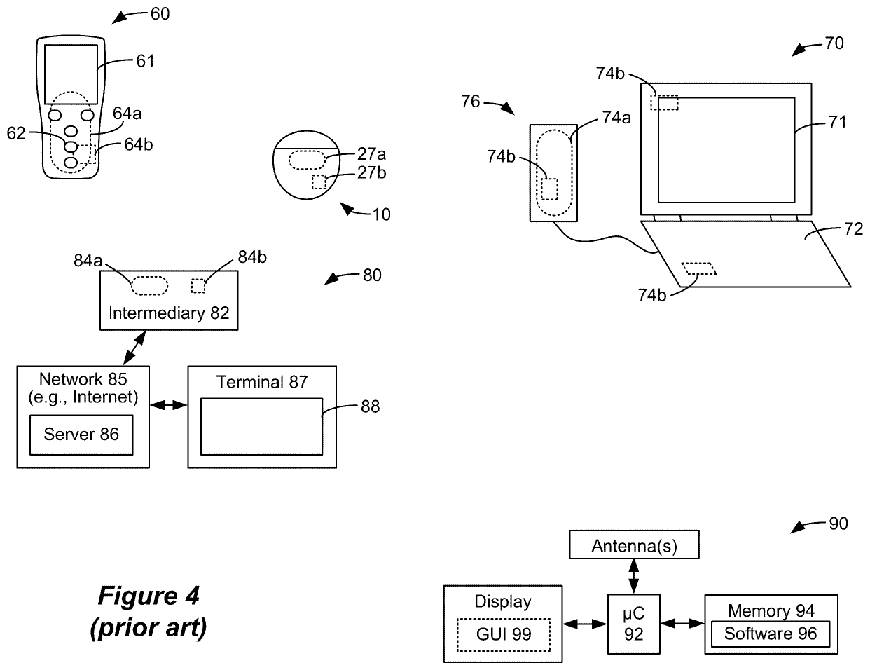

Use of selected electrodes to provide cathodic stimulation sets a particular position for a cathodic pole 120 in three-dimensional space. The position of this cathode pole 120 can be quantified at a particular longitudinal position L along the lead (e.g., relative to a point on the lead such as the longitudinal position of electrode E15), and at a particular rotational angle θ (e.g., relative to the center of electrode E15). Rotation angle θ of the stimulation is typically only relevant when a directional lead such as 19 (FIG. 1C) is used. The position of the cathode pole 120 (−) is shown in a leads interface 102 of the GUI 99, as is the anode pole 121 (+) at the case electrode Ec. Notice that the position of the pole 120 (L,θ) may be virtual; that is, the position may not necessarily occur at the physical position of any of the electrodes 16 in the electrode array, as explained further later. The leads interface 102 preferably also includes an image 103 of the lead being used (e.g., directional lead 19) for the patient. Although not shown, the leads interface 102 can include a selection to access a library of relevant images 103 of the types of leads (e.g., 18 or 19) that may be implanted in different patients, which may be stored with the relevant external system software (e.g., 96, FIG. 4). The cursor 101 can be used to select an illustrated electrode 16, or a pole such as cathode pole 120. Pole 120 could also be anodic (and 120 cathodic), or there could be more than one pole associated with the lead 19 if multipolar stimulation is used, but this isn't shown.

An electrode configuration algorithm (not shown), operating as part of external system's software 96, can determine the position (L,θ) of the cathode pole 120 in three-dimensional space from a given electrode configuration (105), and can also conversely determine an electrode configuration from a given position of the pole 120. For example, the user can place the position of the pole 120 in leads interface 102 using the cursor 101. The electrode configuration algorithm can then be used to compute an electrode configuration that best places the pole 120 in this position. Note in the example shown that the cathode pole 120 is positioned closest to electrodes E8, E9, E11, and E12, and at a longitudinal position between them (i.e., that is between the longitudinal positions of E8 and E9, and Ell and E12). The electrode configuration algorithm may thus calculate based on this position that these electrodes should receive the largest share of cathodic current (20% *−I), as shown in the electrode configuration interface 105. E10 and E13 which are farther away from the pole 120 may receive lesser percentages *10%*−I), again as shown at 105. The electrode configuration algorithm can also operate in reverse: from a given electrode configuration, the position of the pole 120 can be determined and illustrated in the leads interface 102. An electrode configuration algorithm is described further in U.S. Patent Application Publication 2019/0175915, which is incorporated herein by reference.

GUI 99 can further include a visualization interface 106 that allows a user to view a stimulation field image 112 formed on a lead given the selected stimulation parameters and electrode configuration. The stimulation field image 112 is formed by field modelling in the clinician programmer 70, as discussed further in the '091 Publication.

The visualization interface 106 preferably, but not necessarily, further includes tissue imaging information 114. This tissue imaging information 114 is presented in FIG. 5A as three different tissue structures 114a, 114b and 114c for the patient in question, which tissue structures may comprise different areas of the brain for example. Such tissue imaging information may come from a Magnetic Resonance Image (MRI) or Computed Tomography (CT) image of the patient, or a generic library of images, and may include user defined regions. One skilled will realize that the tissue imaging information 114 comprises data retrieved by the external system being used, and may be stored as part of that system's memory 94 (FIG. 4).

The tissue imaging information 114 is preferably registered to the lead 19 such that the position of the lead 19 (and the electrodes) within the tissue imaging information 114 (and hence the patient's tissue) is known. This allows the GUI 99 to overlay the lead image 111 and the stimulation field image 112 with the tissue imaging information 114 in the visualization interface 106 so that the position of the stimulation field 112 relative to the various tissue structures 114i can be visualized. The various images shown in the visualization interface 106 (i.e., the lead image 111, the stimulation field image 112, and the tissue structures 114i) can be three-dimensional in nature, and hence may be rendered to allow such three-dimensionality to be better appreciated by the user, such as by shading or coloring the images, etc. A view adjustment interface 107 may allow the user to move or rotate the images, using cursor 101 for example, as explained in the '091 Publication. In FIG. 5A, a cross-section interface 108 allows the various images to be seen in a particular two-dimensional cross section, and in this example a cross section 109 is shown taken perpendicularly to the lead image 111 and through split-ring electrodes E11, E12, and E13. Interfaces 106 and 108 may also show the position of cathode pole 120, but this isn't shown.

The GUI 99 of FIG. 5A is particularly useful because it allows the electric field as reflected in stimulation field image 112 (or the pole 120) to be seen relative to surrounding tissue structures 114i. This allows the user to adjust the stimulation parameters to recruit, or avoid recruiting, particular tissue structures 114i. Assume for example that it is desirable for a given patient to stimulate tissue structure 114a, but to not stimulate tissue structure 114c. This may be because tissue structure 114a is causing undesired patient symptoms (e.g., tremor) that stimulation can alleviate, while stimulation of tissue structure 114c will cause undesired side effects. Tissue structure 114b may be expected when stimulated to provide neither therapeutic benefits nor significant side effects. The clinician can then use GUI 99 to adjust stimulation (e.g., to adjust the stimulation parameters or the electrode configuration) to move the stimulation field 112 (e.g., the cathode pole 120) to a proper position (L,θ) relative to these tissue structures. In the example shown, and as best seen in the cross-section interface 108, higher cathodic currents are provided at split-ring electrodes E11 and E12 (0.2*−I) because these electrodes are generally facing towards tissue structure 114a which should be stimulated. By contrast, split-ring electrode E13 carries a smaller cathodic current (0.1*−I) because it generally faces towards tissue structure 114b where stimulation would not be expected to be effective. Electrodes proximate to tissue structures 114c are off and carry no current. The result is a stimulation field 112 that is more predominant in tissue structure 114a, less predominant in tissue structure 114b, and avoids tissue structure 114c, as shown in the visualization interface 106.

Whether tissue structures should be stimulated or avoided can depend on a number of factors, such as the patient's diagnosis or the symptoms he is experiencing. For example, a patient with predominant tremor might benefit from stimulating the dorsal part of the subthalamic nucleus (STN) closer to the Internal Capsule, whereas a patient with predominant gait problems might benefit from stimulating the ventral part of the STN closer to the Substantia Nigra, and thus these tissue structures may correspond to tissue structures 114a. One skilled in the art would similarly understand tissues structures that should be avoided when providing DBS stimulation therapy corresponding to tissue structures 114c.

Especially in a DBS application, it is important that correct stimulation parameters be determined for a given patient. Improper stimulation parameters may not yield effective relief of a patient's symptoms, or may cause unwanted side effects. To determine proper stimulation, a clinician typically uses a GUI such as GUI 99 to try different combinations of stimulation parameters. This may occur, at least in part, during a DBS patient's surgery when the leads are being implanted. Such intra-operative determination of stimulation parameters can be useful to determine a general efficacy of DBS therapy. However, finalizing stimulation parameters that are appropriate for a given DBS patient typically occurs after surgery after the patient has had a chance to heal, and after the position of the leads stabilize in the patient. Thus, at such time, the patient will typically present to the clinician's office to determine (or further refine) optimal stimulation parameters during a programming session.

Gauging the effectiveness of a given set of stimulation parameters typically involves programming the IPG 10 with that set, and then reviewing the therapeutic effectiveness and side effects that result. Therapeutic effectiveness and side effects are often assessed by one or more different scores (S) for one or more different clinical responses, which are entered into the GUI 99 of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst).

Scores can also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI 99, and if necessary, converted to a score. U.S. Patent Application Publication 2021/0196956, which is incorporated herein by reference in its entirety, discusses determining which symptoms and/or side effects are most sensible to score for a given patient.

One type of objective measurement proposed for use in DBS systems are Evoked Resonant Neural Activity (ERNA) responses, which are described in U.S. Patent Application Publication 2023/0099390; Sinclair, et al., “Subthalamic Nucleus Deep Brain Stimulation Evokes Resonant Neural Activity,” Ann. Neurol. 83(5), 1027-31 (2018). An ERNA comprises an oscillatory voltage response provided by brain tissue in response to stimulation. Stimulation of the STN, and particularly of the dorsal subregion of the STN, has been observed to evoke strong ERNA responses, whereas stimulation of the posterior subthalamic area (PSA) does not evoke such responses. Thus, ERNA may provide a biomarker for selecting appropriate electrodes for stimulation and for achieving the desired therapeutic response. Having said this, ERNA is just one type of neural potential that can be monitored in response to stimulation. Local field potentials (LFPs), DBS local evoked potentials (DLEPs), evoked compound activity (ECA), single or multi unit activities (SUA/MUA), or even EEG/ECOG may also be monitored and used in the disclosed techniques. Other brain regions which may evoke or not evoke ERNA may also be employed. The '390 Publication discloses an IPG capable of sensing neural potentials such as ERNAs, and discusses algorithms for interpreting and using such sensed data. Further, U.S. Patent Application Publication 2023/0271015 discusses the use of ENRA measurement as scores used in algorithms to assist in the selection of optimal stimulation parameters for a DBS patient.

Once the GUI 99 of the clinician programmer 70 has received various scores S (whether subjective or objective, and whether from patient or clinician) for each of the sets of stimulation parameters tested, the clinician may review the scores to try and determine one or more sets of optimal stimulation parameters for the patient which maximize therapeutic effectiveness while minimizing unwanted side effects. Typically, this process involves significant guess work and time, especially when a directional lead such as 19 (FIG. 1C) is used. If testing all possible stimulation parameter sets is done as comprehensively as possible, stimulation would need to be provided to the patient at every possible stimulation position (L,θ)—as reflected in various electrode configurations—the IPG is capable of producing. Then different combinations of the waveform parameters (e.g., F, PW, and I) would need to be tested at each of these positions, and then scored (S), with potentially more than one score being taken at each location.

In reality, it may only be necessary to optimize the waveform parameter of amplitude (I), as other stimulation parameters (frequency F, pulse width PW) may be known or determined by other means. Nevertheless, at least one score would need to be determined for all possible combinations of I, L and θ the IPG 10 is capable of producing. This may be a burdensome number of combinations to try during a programming session. At any given combination of I, L and θ, it may take some time (e.g., a matter of minutes) to determine an appropriate score (S). This is particularly true if the scores are based on subjective measurements, because the patient may need to be observed, may need to perform certain tasks (finger tapping, walking, etc.), or may need to answer a number of questions. Because a programming session may only reasonably last a few hours, only a fraction of possible I, L and θ combinations can be tested and scored. While scores provided by objective measurements such as those based on ERNA neural response measurements may be quicker to perform, it may still be inefficient to measure such responses at each possible combination of I, L and θ. This makes it difficult to determine optimal stimulation parameters for the patient.

U.S. Patent Application Publication 2022/0257950, which is incorporated by reference in its entirety, discloses an optimization algorithm 200 to efficiently test different I, L and θ combinations with the goal of more quickly arriving at optimal stimulation parameters for a given patient. As stated in the '950 Publication, another waveform parameter (e.g., F, PW) could also be optimized, but the waveform parameter of amplitude (I) is chosen for optimization given its high significance to patient treatment.

This disclosure improves upon the optimization algorithm provided in the '950Publication. U.S. Patent Application Publication 2023/0271015; U.S. patent application Ser. No. 18/427,464, filed Jan. 30, 2024; and U.S. patent application Ser. No. 18/753,832, filed Jun. 25, 2024, also disclose modified versions of algorithm 200, and these applications are also incorporated herein by reference.

Optimization algorithm 200 is shown at a high level in FIG. 6, with other sub-steps and details shown in subsequent figures. The algorithm 200 preferably first simultaneously determines an optimal longitudinal position (Lopt) and amplitude (Iopt1) for stimulation (300) along the lead. When determining Lopt and Iopt1, and assuming a directional lead (e.g., 19, FIG. 1C) is used, stimulation is positioned symmetrically (non-directionally) around the lead during testing by setting currents equal at split ring electrodes at a common longitudinal position. As explained further below, determining Lopt and Iopt1 involves the algorithm 200 efficiently selecting various values for longitudinal position L and amplitude I (L,I) at which stimulation can be tried on the patient and scored. This is an iterative process, and the algorithm 200 automatically determines a next (L,I) value to be tested and scored based on previously tested and scored (L,I) values.

Once optimal longitudinal position Lopt is determined, the algorithm 200 determines whether Lopt is proximate to split ring electrodes (370)—i.e., whether Lopt is longitudinally at or close to split ring electrodes on a directional lead 19. Lopt may be determined to be proximate to split ring electrodes if at least one split ring electrode is used (active) to set the position of Lopt, as explained further below. If Lopt is not proximate to split ring electrodes, as would necessarily be the case when the algorithm 200 is used with a non-directional lead (e.g., 18, FIG. 1B), Lopt and Iopt1 are optimized for the patient (380); optimizing a rotational angle θ is irrelevant and thus no further optimization is required.

If Lopt is proximate to split ring electrodes (370), as might be the case when algorithm 200 is used with a directional lead 19, the algorithm 200 simultaneously determines an optimal rotational angle (θopt) and amplitude (Iopt2) for stimulation around the lead at the optimized longitudinal position Lopt (400). Algorithm 200 may determine that Iopt2 is the same as Iopt1 determined earlier, but it is also likely that Iopt2 will differ from Iopt1 as the rotational angle of stimulation is also optimized. As explained further below, determining θopt and Iopt2 involves the algorithm 200 selecting various values for rotational angle θ and amplitude I (θ,I) which can be tried on the patient and scored. This process can be similar to the manner in which (L,I) values were selected earlier, with the algorithm 200 automatically and efficiently determining next (θ,I) values to be tested and scored based on previously tested and scored (θ,I) values. Once θopt and Iopt2 are optimized, the stimulation is fully optimized for the patient, as the longitudinal position, rotational angle, and amplitude of the stimulation (Lopt, θopt, Iopt2) have now been determined (450).

One skilled in the art will appreciate that programming algorithm 200 can comprise a portion of software 96 operable in the clinician programmer 70 or other external system (FIG. 4). Algorithm 200 can be stored as instructions on a computer-readable medium, such as on a magnetic or optical disk, in solid state memory, etc., and may be so stored in the clinician programmer 70 or in any external system (see FIG. 4).

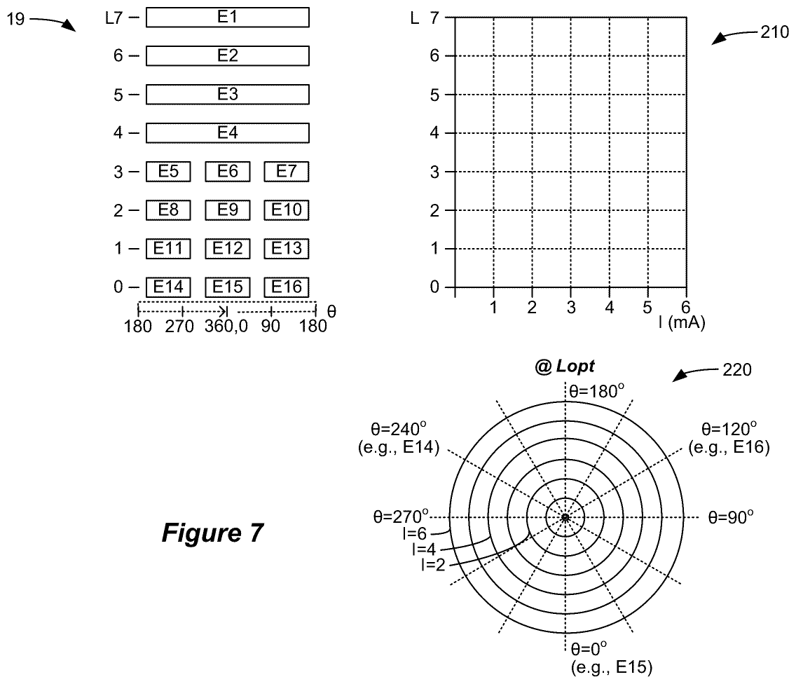

FIG. 7 shows various representations that are useful in understanding the further description of programming algorithm 200 that follows. First, a directional lead such as 19 (FIG. 1C) is illustrated in a two-dimensional manner. This directional lead 19 is shown as a more complicated case, but as noted above algorithm 200 may also be used to determine optimal stimulation parameters when a non-directional lead (e.g., 18, FIG. 1B) is used. As shown, this directional lead 19 includes ring electrodes E1-E4 which are respectively located at longitudinal positions (L) 7, 6, 5, and 4, and split ring electrodes E5-E16 which are located at longitudinal positions (L) 3, 2, 1, and 0 as shown. L can also be represented by an actual physical measurement, such as millimeters. Rotational angle θ around the lead 19 is also shown, with 0 degrees being selected at some arbitrary position (such as in the middle of split ring electrodes E6, E9, E12, and E15). Of course, this type of directional lead 19 is just one example, and other directional leads having different combinations of ring and split ring electrodes, or directional leads having split ring electrodes exclusively, could be used as well.

L,I parameter space 210 shows possible values (L,I) that can be tested and optimized, which is particularly useful during step 300 (FIG. 6) when Lopt and Iopt1 are determined. L,I parameter space 210 can have any resolution that the IPG 10 is capable of producing. For example, it can be assumed in one example that the amplitude I of the current is adjustable in increments of 0.1 mA, up to a maximum of 6.0 mA (from 0.0 to 6.0 mA). Longitudinal position L may also be set in increments of tenths (from 0.0 to 7.0).

θ,I Parameter space 220 shows possible values for (θ,I) that can be tested and optimized, which is particularly useful during step 400 (FIGS. 6, 12, 13) when θopt and Iopt2 are determined at the already-established position of Lopt. θ,I parameter space 220 as shown is circular, with rotational angle θ represented angularly, and with amplitude I represented radially. Parameter space 220 can again have any resolution that the IPG 10 is capable of producing. For example, it can be assumed in one example that the rotational angle θ is adjustable in increments of 30° degrees, and again the amplitude I of the current is adjustable in increments of 0.1 mA up to a maximum of 6.0 mA.

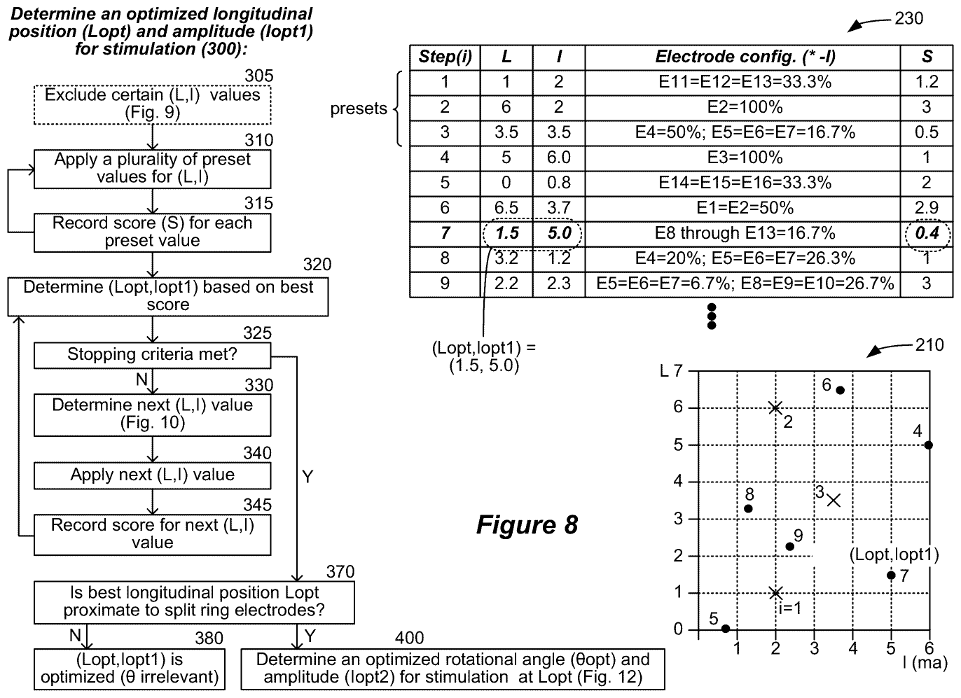

FIG. 8 shows steps involved in determining Lopt and Iopt1 (300), which involves an iterative testing of various (L,I) values. See also U.S. Patent Application Publication 2018/0104500, which is incorporated by reference in its entirety.

In a first optional step 305, certain (L,I) values in the L,I parameter space 210 may at the outset be excluded from further consideration and testing in the algorithm 200. Such exclusions are useful to reduce the number of (L,I) values that must be assessed and potentially tested by the algorithm 200, reducing computational complexity. Exclusion of (L,I) values may be manual (by using input from the clinician into the GUI 99), or automated, or semi-automated, in the algorithm 200. Furthermore, some amount of cursory patient testing—providing stimulation at various longitudinal positions L and/or amplitudes I—can be useful at step 305 in generally determining and inputting into the algorithm 200 (L,I) values that should be excluded.

Exclusions at step 305 may occur for a number of different reasons. First, stimulation provided at certain (L,I) values may simply be understood (e.g., based on testing) as unlikely to provide therapeutic effectiveness. Thus, stimulation at particular longitudinal positions L along the lead 19 may simply be too far away from tissue structures of interest. Certain amplitude values I the IPG can produce may simply be too low or too high to be expected to be useful. (L,I) values corresponding to these longitudinal positions and amplitudes may therefore simply be excluded.

Exclusion of certain (L,I) values may also occur step 305 based on objective testing. In a preferred example, such objective testing can comprise measuring neural potentials (e.g., ERNAs) discussed earlier. This was discussed in detail in the above-incorporated '015Publication.

Exclusion of certain (L,I) values may also occur step 305 based on an analysis of the surrounding tissue structures, which may involve the use of tissue imaging information 114, as discussed earlier with reference to FIG. 5A. For example, (L,I) values proximate to tissue structures that should not be stimulated may be excluded. This is discussed in further detail in the above-incorporated '464 Application.

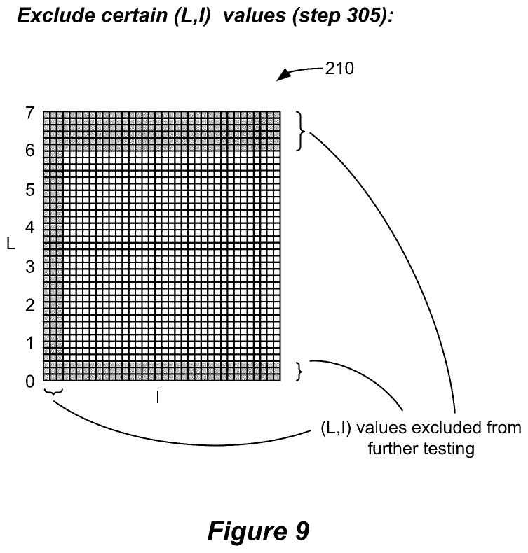

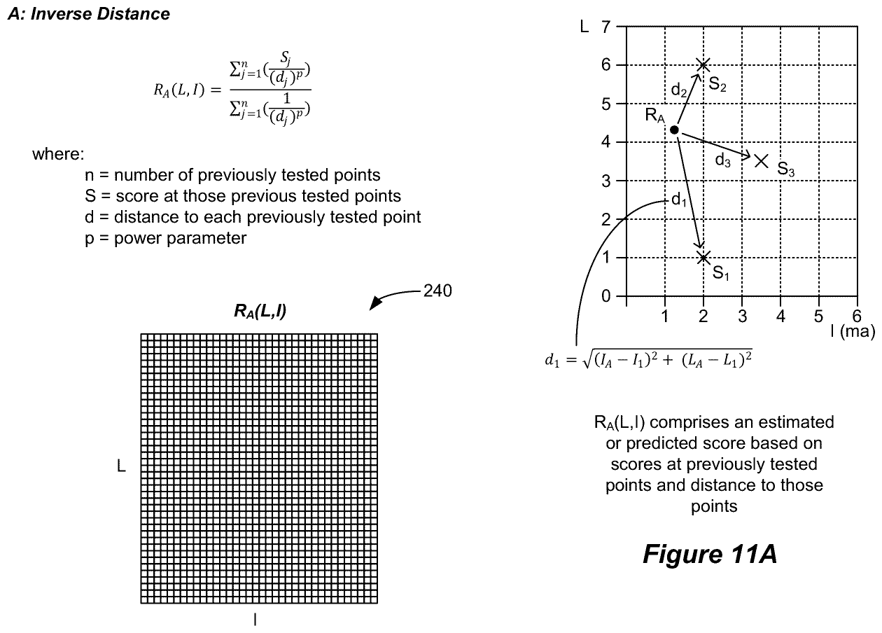

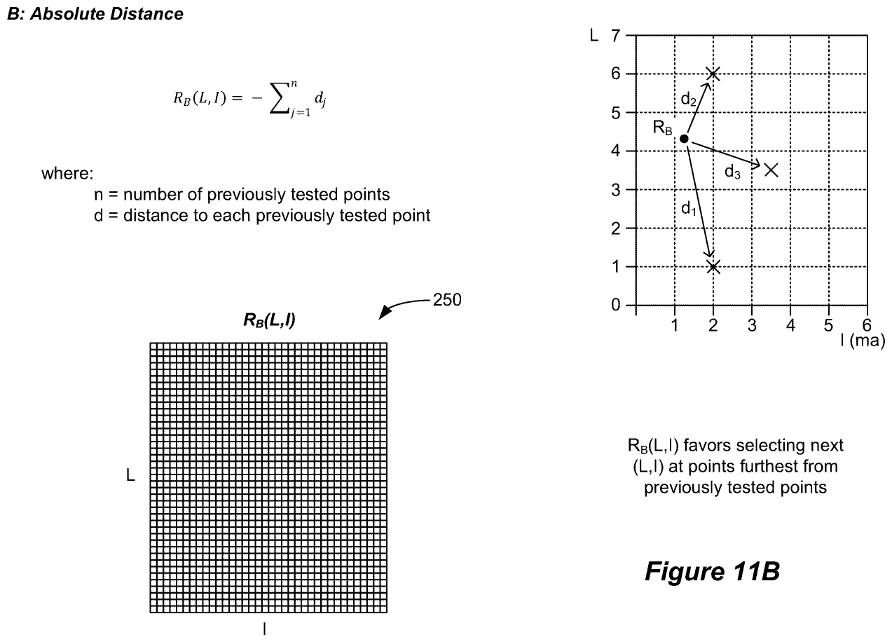

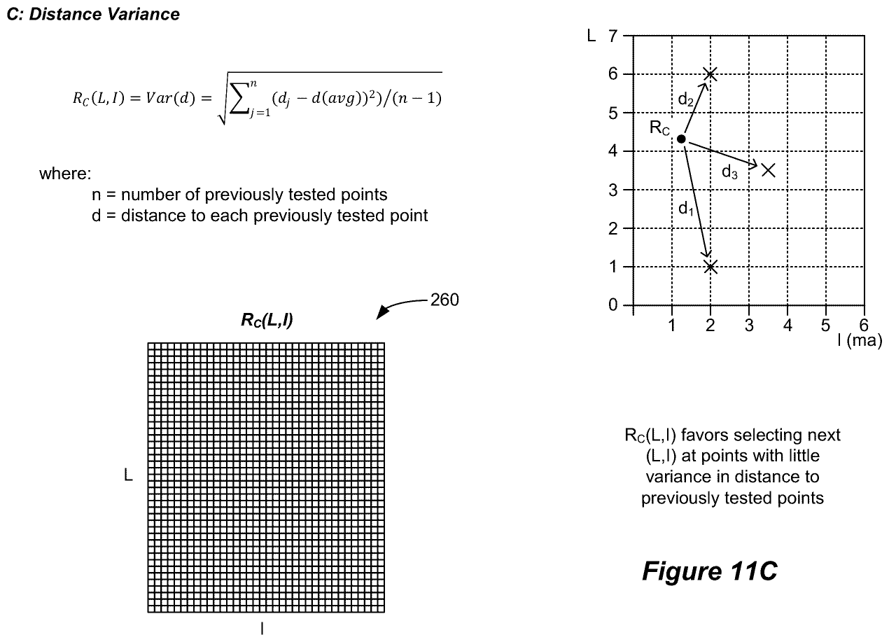

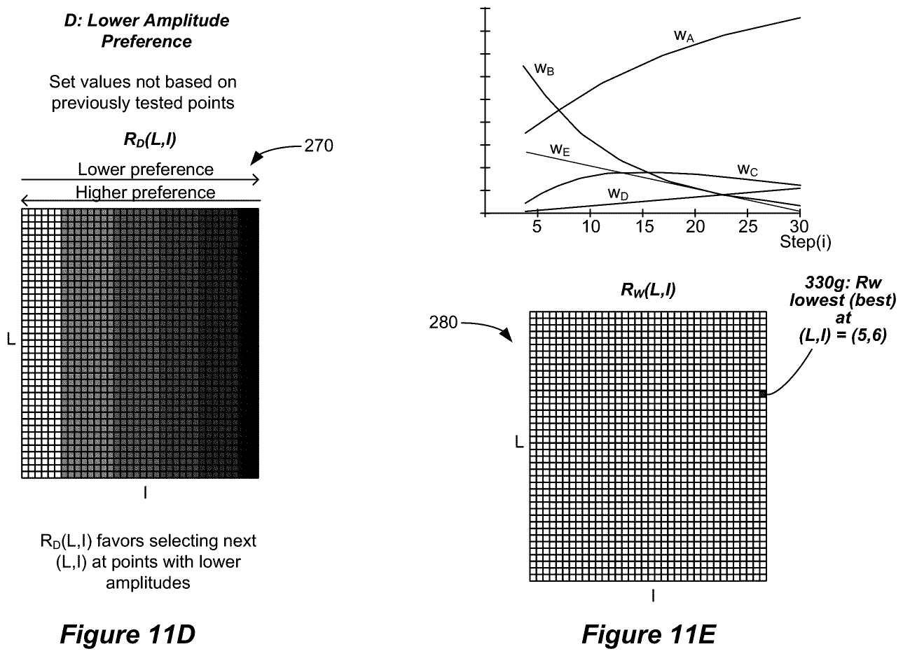

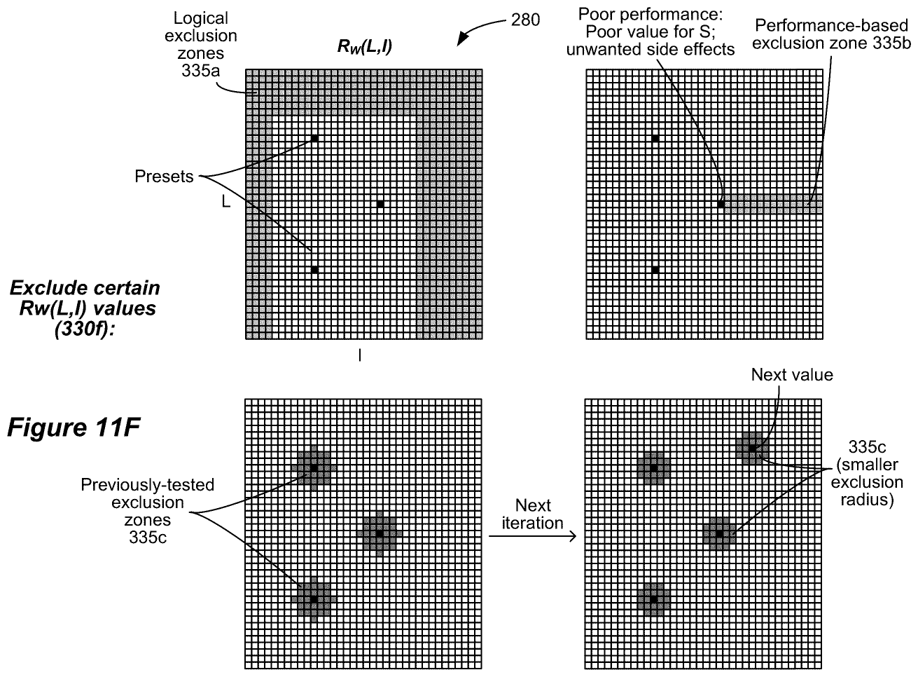

FIG. 9 generally shows the effect of excluding certain L,I values from consideration during operation of the algorithm 200. In FIG. 9, various (L,I) values with a low amplitude (e.g., 0<I<0.5 mA) have been excluded (presumably because they are expected to be too low to be effective), as well as various (L,I) values at certain longitudinal positions (e.g., 0<L<0.5; 6<L<7) (perhaps because they are too far away from desired tissue structures 114a, too close to tissue structures 114c that should be avoided, or otherwise generally ineffective). Regardless of the reasons, exclusion of (L,I) values at step 305 cases the amount of data that the algorithm 200 must process, which increases the likelihood that algorithm 200 will quickly and accurately discover optimal stimulation parameters for the patient. Still further exclusions may be made during the operation of the algorithm 200, as explained later (see FIG. 11F).

Referring again to FIG. 8, the algorithm 200 preferably uses a plurality of preset (L,I) values at its inception (310). These preset values will depend on the type of lead for which stimulation is being optimized, and are preferably chosen to cover a desired portion of the (non-excluded) L,I parameter space 210, and/or the most probable position of optimal stimulation. In the example shown, three presets are used with (L,I) values of (1, 2 mA), (6, 2 mA) and (3.5, 3.5mA), which are sequentially selected in different steps (i=1, 2, and 3) and applied to the patient. As explained further below, applying (L,I) values involves the clinician programmer 70 transmitting stimulation parameter sets (as reflected in the electrode configuration) to the IPG 10 so that stimulation can be produced at the prescribed position (L) and amplitude (I). If one or more of these (L,I) presets has been excluded (305, FIG. 8), it can be relocated by the algorithm 200 to another (L,I) value in the L,I parameter space 210, although this detail is not shown. Furthermore, the presets can be relocated to other (L,I) values depending on the results of objective testing at step 305.

A data set 230 is formed in the clinician programmer 70 as the algorithm 200 runs, and includes the electrode configurations necessary to form stimulation at the prescribed longitudinal positions, L. For example, and referring to illustration of lead 19 in FIG. 7, notice that longitudinal position L=1 (step i=1 for the first preset value) corresponds to the location of split ring electrodes E11, E12, and E13. Because step 300 only seeks to determine L and I without imparting any directionality (rotation) to the stimulation (i.e., such that the stimulation field 112 is symmetric around the lead), these electrodes E11, E12, and E13 will share the cathodic current equally (33%*−I), effectively placing cathodic pole 120 longitudinally at L=1. More specifically, because I=2 mA at this step i=1, each of electrodes E11, E12, and E13 will receive −0.67 mA at this step (or as close as possible to this, given the IPG's resolution. For example, if the IPG 10 can provide current in 0.1 mA increments, each of the electrodes E11, E12, and E13 may receive −0.7 mA).

Longitudinal position L=6 (step i=2 for the second present value) corresponds to the location of ring electrode E2, which will receive 100% of the cathodic current (100%*−I) to place cathode pole 120 longitudinally at this position. More specifically, because I=2 mA at this step i=2, electrode E2 will receive −2.0 mA at this step.

Longitudinal position L=3.5 (step i=3 for the third preset value) is directly between ring electrode E4, and the longitudinal position of split ring electrodes E5, E6, and E7. Therefore, to place the cathode pole 120 (virtually) as this position, the cathodic current is shared equally between E4 (50%*−I) and E5, E6, and E7 as a group (with each receiving 16.7%*−I). More specifically, because I=3.5 mA at this step i=3, each of electrodes E5, E6, and E7 will receive-0.6 mA at this step (rounded), with E4 receiving −1.7 mA. Although not shown, remember that these electrode configurations as reflected in data set 230 are determinable in the external system software 96 using the electrode configuration algorithm described earlier, which can comprise a portion of optimization algorithm 200.

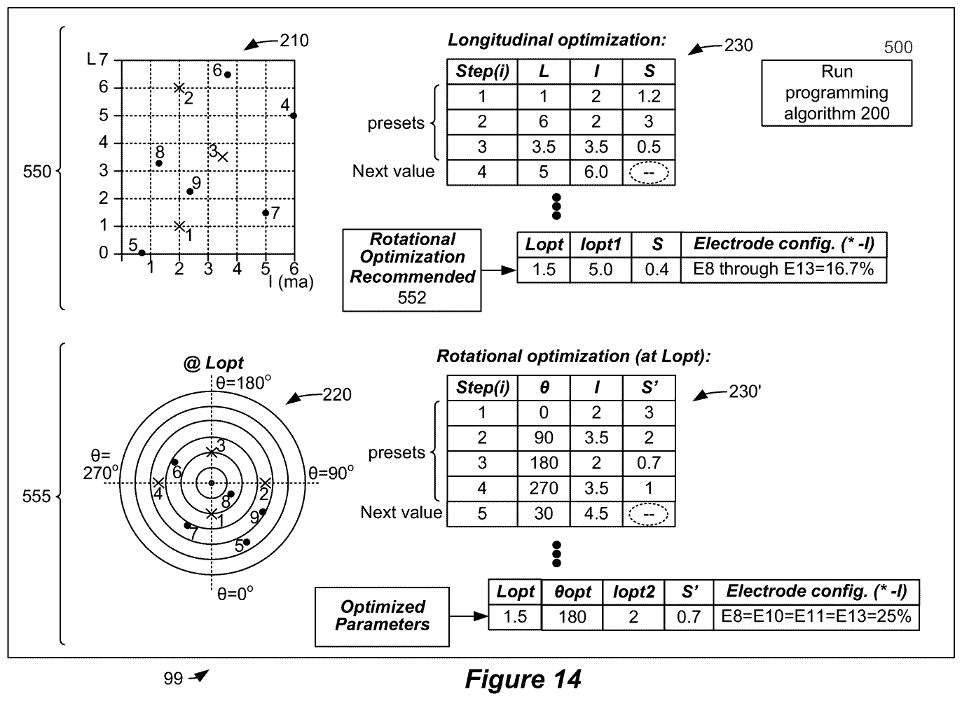

The stimulation parameters as embodied in the (L,I) presets and as determined by the electrode configuration algorithm (the active electrodes; whether they are anodes or cathodes, and the amplitude at each active electrode) are sequentially transmitted to the patient's IPG 10 (along with other non-optimized parameters such as frequency F and pulse width PW) so that the stimulation can be applied to the patient. As each of these stimulation parameters sets are applied, at least one score (S) is then determined for each (315). As noted above, a score can comprise any metric (subjective or objective) that indicates therapeutic effectiveness of and/or the side effects resulting from the stimulation parameters sets. As assumed earlier, a lower score in the depicted example indicates a better result, with 0 being good and 4 poor, although a different scale could be used in which higher numbers are better. The scores (S) once determined for each of the presets are entered into the data set 230 in the clinician programmer 70, such as by having the clinician type the score into the GUI 99 (see FIG. 14). Scores can also automatically be populated into the data set 230 if they are objectively measured and determined, as discussed in the '015Publication incorporated earlier. In FIG. 8, notice that the first (L,I) point (1,2) results in a score of 1.2; the second (6,2) results in a score of 3 (poor); and the third (3.5, 3.5) results in a score of 0.5 (good). The score S can comprise a composite based on a number of metrics that indicate effective and/or side effects, and a plurality of scores can also be determined at each (L,I) value, but this detail is not yet shown for simplicity.

After sequentially applying stimulation according to these presets and determining and recording their scores S after patient testing, the optimization algorithm 200 can determine a best of the (L,I) values (Lopt, Iopt1) based on the scores at those points (320). As explained further below, as the algorithm 200 iterates, more (L,I) values will be tested and scored, and (Lopt, Iopt1) can be updated accordingly at this step. At this point, after only testing the presets, (Lopt, Iopt1) is determined at step 320 to be (3.5,3.5), because this tested value yields the best (e.g., lowest) score (of 0.5).

Next, the algorithm 200 determines whether one or more stopping criteria have been met (325). If a stopping criterium has been met (325), the algorithm 200 may stop iterating--i.e., stop determining and testing further (L,I) values-at which point (Lopt, Iopt1) can be established. Any number of stopping criteria can be used. For example, the algorithm 200 may decide to stop: if a last determined (L,I) value is too close to other values that have been tested; if the scores at a number of preceding (L,I) values are poor (suggesting that the algorithm is no longer suggesting new (L,I) values to useful effect); if a score at the last selected value is significantly good (e.g., 0, suggesting that the algorithm can simply select this last value as the optimal point); if a maximum number of steps (i) has been reached; etc. The stopping criteria need not be automated in the algorithm 200. For example, a stopping criterium may simply comprise the clinician deciding that a suitable number of (L,I) values has been tested and no further steps are required.

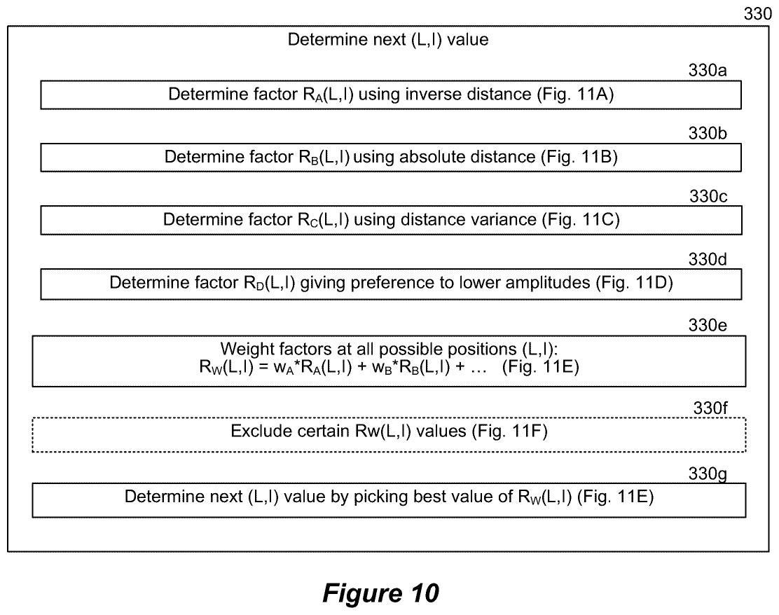

If a stopping criteria has not been met (325), the algorithm 200 proceeds to determine a next (L,I) value to be tested (330) in a next iteration (step i=4). Details involved in choosing this next (L,I) value are shown first with respect to FIG. 10. The algorithm 200 computes and considers one or more factors, and the illustrated example considers four factors R_{A}, R_{B}, R_{C}, and R_{D}) (330a-d), although more or fewer factors could be used as discussed further in some of the references incorporated above. Each of these factors is preferably calculated at all possible (L,I) points in the L,I parameter space 210 that weren't excluded earlier (FIG. 8, step 305). As explained below, factors R may be determined based on the distance to all previously-tested (L,I) values, the scores S at those points, or based on other considerations explained further below. When more than one factor is used, the factors can be weighted (which can include normalizing or ranking) and summed (R_{W}) at each (L,I) value (330e), as explained further below. Certain (L,I) values can also later be excluded (330f), as explained further below. A next (L,I) value to be tested can be determined by picking the weighted factor R_{W}(L,I) having the best (e.g., lowest) value (330g), again as explained in further detail below.

Step 330a calculates factor R_{A} for all (L,I) positions using an inverse distance metric, as shown in FIG. 11A. The calculated value for R_{A} at each (L,I) position in L,I parameter space 210 is represented by data set 240, which is determined and stored in the clinician programmer 70 (or other external system) as the algorithm 200 runs. Note that the number of entries comprising R_{A} data set 240 depends on the resolution at which both I (e.g., 0.1 mA) and L (e.g., in tenths) are defined in the system, as well as the extent to which L,I parameter space 210 is searched (some values may have been excluded earlier. See FIG. 8, step 305). The equation for calculating R_{A} at each (L,I) point in data set 240 is shown in FIG. 11A. Note from this equation that factor R_{A} relies on both the previous position of previously-tested (L,I) values (in steps i=1-3) as reflected in the inverse distance 1/dⱼ from each (L,I) point to the previously-tested points. R_{A} also relies on the scores Sᵢ (S₁, S₂, S₃) at each of those previously-tested points. In this example, (L,I) values in data set 240 having lower values for R_{A} are more likely to selected as a next (L,I) value to test, as explained further below.

Note as shown in the equation in FIG. 11A that distance d can be determined in a Euclidian fashion, and can comprise the square root of the sum of the differences (in L and I) squared. However, distance could be computed in other fashions. For example, stimulation field modelling (SFM) can be used to model a volume of activation (VOA) in the tissue both at a given (L,I) point and each of the previously-tested points, with a centroid or some other relevant point within those VOA used to determine the distances. SFM modelling may also be beneficial to determining whether stimulation will overlap with an exclusion zone, as discussed further below with respect to FIG. 11F. Variable ‘p’ represents a power parameter that tends to accentuate the distances dⱼ and can be empirically set (e.g., to 5 in one example). In effect, factor R_{A} comprises an estimated or predicted score at other (L,I) values that have not yet been tested, and such estimated or predicted scores are based upon the scores at previously-tested positions, as well as the inverse distances to those points. Note that actual calculated values for R_{A} at each (L,I) point are not shown in data set 240. Different prediction-based calculations could be used to determine factor R_{A} as well.

Returning to FIG. 10, step 330b determines a second factor R_{B} for all (L,I) positions using an absolute distance metric, as shown in FIG. 11B. This factor, generally speaking, tends to favor selection of a next (L,I) point that is furthest away from previously-tested (L,I) points, and is thus beneficial in that it encourages the algorithm 200 to select a next (L,I) point for testing at locations in L,I parameter space 210 that haven't yet been tested. The calculated value for R_{B} at each position (L,I) is represented by data set 250, which is determined and stored in the clinician programmer 70 as the algorithm 200 runs. The equation for calculating R_{B} at each (L,I) point in data set 250 is shown in FIG. 11B, and comprises the sum of the distances dⱼ from each (L,I) point to the previously tested points. Because it is desired that lower values for R_{B} are preferred when selecting a next (L,I) value to be tested in the depicted example, this sum is made negative, such that (L,I) values with longer summed distances to previously-tested points are more likely to be selected. However, other means can be used to translate larger summed distances into lower values for R_{B} (e.g., by taking their inverse). Note that R_{B}, unlike R_{A}, does not rely on the scores Sᵢ at previously tested points. Again, actual values for R_{B} at each (L,I) point are not shown in FIG. 11B.

Returning to FIG. 10, step 330c determines a third factor R_{C} for all (L,I) positions using a distance variance metric, as shown in FIG. 11C. This factor, generally speaking, tends to favor selection of a next (L,I) point that is most equidistant from previously tested (L,I) points. The calculated value for R_{C} at each position (L,I) is represented by data set 260, which is determined and stored in the clinician programmer 70 as the algorithm 200 runs. The equation for calculating R_{C} at each (L,I) point in data set 240 is shown in FIG. 11C, and simply comprises the variance of the distances dⱼ from each (L,I) point to the previously tested points, with lower values for R_{C} indicating (L,I) points more likely to be selected as the next (L,I) value to be tested. Note that R_{C}, like R_{B}, does not rely on the scores Si at previously tested points. Again, actual values for R_{C} at each (L,I) point are not shown in FIG. 11C.

Returning to FIG. 10, step 330d determines a fourth factor R_{D} for all (L,I) positions using a preference for lower amplitudes, as shown in FIG. 11D. This factor, generally speaking, tends to favor selection of a next (L,I) point that has lower values of amplitude, I. This factor is reasonable to consider as it is generally preferred to provide a patient stimulation that is as low in amplitude as possible. The values for R_{D} at each position (L,I) is represented by data set 270. This data set 270 may be preset and not based on the position of or scores at previously tested values. For example, R_{D} may be higher at (L,I) points having higher amplitudes (e.g., R_{D})=4 when I>5 mA) and lower at (L,I) values having lower amplitudes (e.g., R_{D})=0 when I<1 mA). These preset values are shown in data set 270 using shading, with more-preferred lower amplitudes having a lighter shading, and less-preferred higher amplitudes having a higher shading.

Other factors Ri may be considered as well, but are not illustrated here. For example, another factor can be determined using a preference to recruit particular tissue structures, as discussed further in the above-incorporated '464 Application. Algorithm 200 doesn't require the use of all of the factors described, and still other factors that aren't shown could be used as well. These factors could also be computed differently.

Returning to FIG. 10, a weighted factor R_{W} for all (L,I) positions is determined using the factors R_{A}, R_{B}, R_{C}, and R_{D} determined earlier (330e). In one example, weights W_{A}, W_{B}, W_{C}, and W_{D} can be multiplied by their associated factors (in data sets 240-270), and summed to yield R_{W}(L,I)=W_{A}*R_{A}(L,I)+W_{B}*R_{B}(L,I)+W_{C}*R_{C}(L,I)+W_{D}*R_{D}(L,I). The resulting values for R_{W} at each position (L,I) is represented by data set 280 as shown in FIG. 11E.

The weights W applied to the factors can be varied based on user preferences, and FIG. 11E shows example values. FIG. 11E also shows that the weights W can vary in accordance with the step number i—i.e., how many times the algorithm 200 has iterated to determine a next (L,I) value to test, as explained further below. For example, when initially determining a fourth (L,I) value to test after testing the three presets (at step i=4), it may be useful to emphasize factor R_{B}—absolute distance (FIG. 11B)—when determining R_{w} because this factor favors choosing a next (L,I) value that is furthest from the previously-tested presets in the L,I parameter space 210. During later iterations and after more (L,I) positions have been determined and scored, it may be more useful to emphasize factor R_{A}—inverse distance (FIG. 11A)—because this factor places greater weight on the scores determined at the previously-tested (L,I) values. Thus, in FIG. 11E, weight W_{A} is lower and weight W_{B} is higher at earlier iterations of the algorithm 200, but W_{A} is higher and W_{B} is lower at later iterations. Although not illustrated, weights W can also be dependent on recorded scores, or in accordance with other static or dynamic factors.

Note that weighting of the factors to arrive at R_{W}(L,I) can involve some amount of processing of the individual factors R_{I}(L,I). In this regard, note that each of the individual factors R_{I}(L,I) may be of different magnitudes, depending on how such factors are computed. As such, it may be beneficial to normalize the different factors R_{I}(L,I) so that their magnitudes are generally equated before these factors are weighted by weights W_{I}. Alternatively, the weights W_{I} themselves may be adjusted to accomplish such normalization, so that the individual contributions provided by W_{I}*R_{I}(L,I) leading to R_{W}(L,I) are generally equal in magnitude. In another alternative, each of the (L,I) values for a given factor R_{I} can be ranked, with for example a best (lowest) value (L,I) being given a best (e.g., lowest) ranking (e.g., 1), and a worst (highest) value (L,I) being given a worst (highest) ranking (e.g., L*I). Ranking each (L,I) value for each factor RI before weighting tends to normalize the values of each of the factors, making their weighting by W_{I} more meaningful.

Returning to FIG. 10, certain R_{W}(L,I) values can be excluded from the R_{W}(L,I) data set 280 (330f) prior to selecting a next (L,I) value to be tested (330g), and this is explained further with reference to FIG. 11F. Specifically, FIG. 11F shows different examples of (L,I) values that can be excluded from data set 280 and the rationale behind their exclusion at this point in the algorithm 200. Generally speaking, exclusion prevents the algorithm 200 from selecting (L,I) values to be tested that are likely not helpful in determining optimal values Lopt and Iopt1. Exclusion may involve the algorithm 200 applying different exclusion zones 335 that exclude different ranges of (L,I) values. Exclusion zones may be automated via operation of the algorithm 200, or the algorithm 200 may allow the clinician to manually define exclusions zones. For example, although not shown, the GUI 99 may allow the clinician to define one or more exclusions zones. As discussed further below, the exclusion zones my change depending on the iteration (step) of the algorithm 200, and thus such zones 335 may exclude different ranges of values from step to step, including in later iterations (L,I) values that were excluded earlier.

The upper left example of the R_{W}(L,I) data set 280 shows different examples of exclusion zones 335a, which comprises zones of (L,I) values that are logical to exclude from testing for one reason or another. Such exclusions zones 335a may be based on the same factors considered earlier at step 305 (FIG. 8), but implemented at this point in the process. Exclusions zones 335a may also at this point be based on data taken during the algorithm 200. For example, exclusions zones 335a may simply comprise ranges of (L,I) values having poor (high) weighted scores at this point in the process.

The upper right of FIG. 11F shows another example in which a poor score at a previously tested value (shown here as one of the presets) is used to define an exclusion zone 335b. In this instance, it may not be useful to consider other (L,I) values at even higher-amplitude currents, as it might be expected that higher amplitudes will make the performance or side effect worse. Thus, the algorithm 200 automatically, or the clinician manually, may define a performance-based exclusion zone 335b. In this example, exclusion zone 335b excludes all (L,I) values with higher amplitudes from the poor performance value. Exclusion zone 335b also for guard band has excluded some higher-amplitude longitudinal positions around this value as well (at slightly different longitudinal positions from the poor-performing point).

Exclusion zones may 335c be also placed around already-tested (L,I) values, as shown at the bottom left of FIG. 11F. This excludes the algorithm 200 from selecting a next (L,I) value that is at or close to a previously-tested (L,I) value. This is shown first in FIG. 11F relative to the three preset values, with the algorithm 200 excluding (L,I) values that are within a certain distance (e.g., radius) of the presets. The reason for doing so is that the presets have already been tested and scored, and it is therefore not useful at this point for the algorithm 200 to potentially recommend a next (L,I) value to be tested that is close to the presets. Instead, to encourage the algorithm 200 to explore values in the L,I parameter space 210 more distant from the presets, such close (L,I) values are excluded.

As explained further below, the algorithm 200 will eventually iterate to select a new (L,I) value to test and score, as shown at the bottom right of FIG. 11F. This can result in the addition of new exclusion zones, or the modification of previously-determined exclusion zones to now re-include (L,I) points that were previously excluded. For example, after a next (L,I) value is determined and scored, there are now four (L,I) points that have been tested (including the original three presets). Notice that the exclusion zones 335c around each of these points have a reduced radius. This can be useful because while the algorithm 200 generally seeks to select distant next (L,I) values as it iterates, it also does not want to ignore potential (L,I) values of interest that were excluded in earlier iterations simply because these values were close to already tested values. As such, and generally speaking, the exclusion zones may be modified by the algorithm 200 as it iterates, and the shape and size of such zones may change based on step number, i.

To summarize, the algorithm 200 can apply various exclusion rules 330f to exclude one or more less-meaningful values to prevent such values from being next selected for testing, at least during a next iteration. FIG. 11F shows just some examples of exclusion, but other exclusion rules could be used by the algorithm as well. Note that exclusion of (L,I) points can also occur at different points during the algorithm 200. For example, certain (L,I) values could have been excluded when the data sets 240-275 for the individual factors R_{A}-R_{D} were determined in steps 330a-330d.

Although not shown in FIGS. 11A-11F, any of the data sets 240-280 can be displayed to the clinician on the GUI 99. In a useful example, the values of the data at each of the (L,I) values can be mapped to a color, thus allowing the data sets 240-280 to appear as “heat maps” whereby data values and general trends can easily be seen in the data. The ability to view heat maps could be added for example to the GUI shown in FIG. 14, discussed later.

Referring to FIG. 10, once R_{W} is determined at each of the (L,I) values, perhaps with some (L,I) values excluded (305, 330f), a best R_{W}(L,I) value is selected (330g), which determines the next (L,I) value to be tested (330). The best R_{W}(L,I) value can be determined by the algorithm 200 upon reviewing the non-excluded various values for R_{W} in the data set 280, and selecting the (L,I) value associated with that best (e.g., lowest) value. (Again, and depending on how the factors are processed, a best R_{W}(L,I) value may also have a highest value). In the example of FIG. 11E, the lowest R_{W}(L,I) value is assumed to occur at value (5.0, 6.0), which then comprises the next (L,I) value to be tested (at step i=4). Note that if there is more than one best R_{W} value in data set 280 (e.g., two or more R_{W} values having the same lowest value), the algorithm 200 can be programmed with tie-breaking rules to arrive at a single next (L,I) point. For example, the algorithm 200 may prefer to pick a next (L,I) value that has a lowest amplitude, or that is furthest from all previously tested values, etc.

Note that prior data determined upon testing of the patient can be used in place of, or can comprise, a preset value. Further, presets do not necessarily need to be pre-established at set (L,I) points. Instead, the clinician can simply start testing at a particular (L,I) value, record a score, etc. Eventually, when the algorithm 200 has received enough scores at previously-tested (L,I) values, it can begin to automatically determine next values at step 330, and the algorithm can begin to iterate.

Referring again to FIG. 8, once the next (L,I) value has been determined (330), that value (5.0, 6.0) can be populated into data set 230 as a next step (i=4) and thus as a next point to be applied to the patient (340) and scored (345). Again, the electrode configuration algorithm described earlier can determine an electrode configuration suitable to position stimulation at this new longitudinal position. For example, at new (L,I) value (5,6), notice that a ring electrode E3 is located at L=5.0 (FIG. 7), and so that electrode alone will receive all of the cathodic current (100%*−I, or −6.0 mA when I=6.0 is also considered). Once the electrode configuration is determined, stimulation parameters corresponding to this next (L,I) value are transmitted to the IPG 10 and applied to the patient (340) similarly to what was described earlier for the preset (L,I) values. At least one score (e.g., S=1) is then determined for stimulation occurring at this new (L,I) value (345) and input into the data set 230 as described earlier. This score S as before can be based on subjective or objective measurements.

Although not shown, the algorithm 200 may ramp over time (gradually change) the stimulation applied between the different iterations (i.e., between i=1 and 2, i=2 and 3, etc.) so that the change in stimulation applied to the patient (in particular, changes of amplitude) is not abruptly changed in a manner that may cause the patient to suddenly experience discomfort or side effects. Whether or how the algorithm 200 ramps the stimulation parameters between various iterations of the algorithm can be controlled by user selections in GUI 99 (not shown).

At this point, the algorithm 200 can return to step 320, where a best of the tested values (Lopt, Iopt1) is determined and/or updated. As described earlier, this involves looking at the scores associated with each of the previously-tested points (in steps i=1 to 4 to this point). The best (e.g., lowest) of these scores (0.5) is still associated with step i=3 at this point, and so (Lopt, Iopt1) remains (3.5, 3.5), which is not updated.

As the algorithm 200 continues, it again determines if one or more stopping criteria have been met (325). Assuming this doesn't occur, the algorithm 200 determines a next (L,I) value to be tested (330) in a next iteration of the algorithm (step i=5). Determining this next (L,I) value preferably occurs as described earlier by determining factors (R_{A}-R_{D}) at all points (L,I) (excepting excluded points). However, notice that there are now more previously-tested points (L,I) to consider (i.e., four, instead of the initial three presets), meaning that the sums in the equations shown in FIGS. 11A-11C involve additional terms (n=4). Again, the factors R_{A}-R_{D} are weighted to determine R_{W}(L,I), and a next (L,I) value is selected (0, 0.8) for testing using a best value from R_{W} (see data set 230, FIG. 8).

This next (L,I) value is applied (340) and scored (345) (S=2); (Lopt, Iopt1) is determined and possibly updated (320), etc. The effect of such successive iterations of the algorithm 200 is shown in the data set 230 and the L,I parameter space 210 of FIG. 8, which shows the (L,I) values that were tested at each iteration (i) of the algorithm 200, and how they logically and efficiently traverse the L,I parameter space 210 as dictated by factors R_{A}-R_{D} and their weightings. Note that the data shown in FIG. 8 is fictitious and only provided to help illustrate operation of the algorithm 200.

Once a stopping criterium has been met (325), an optimal value of (L,I) result (Lopt, Iopt1) as determined and updated earlier during step 320. In the illustrated example of the data set 230 in FIG. 8, it is assumed that the algorithm went through nine iterations (step i=9) before reaching a stopping criterium. Review of the scores at each of the tested (L,I) values yields a best (lowest) score of 0.4, corresponding to Lopt=1.5, Iopt1=5.0 mA (step i=7). More specifically, this corresponds to optimal stimulation parameters of Iopt1=5.0, which is shared equally (as a cathodic current) by each of electrodes E8 E13 (see data set 230), each of which receives a current of 16.7% * −5.0, or −0.83 mA (this current may be rounded to what the IPG 10 is capable of producing). This produced stimulation at the correct longitudinal position (Lopt) and at an optimal amplitude. The pulse width (PW) and frequency (F) used to provide the pulses during operation of the algorithm may also comprise optimal stimulation parameters.

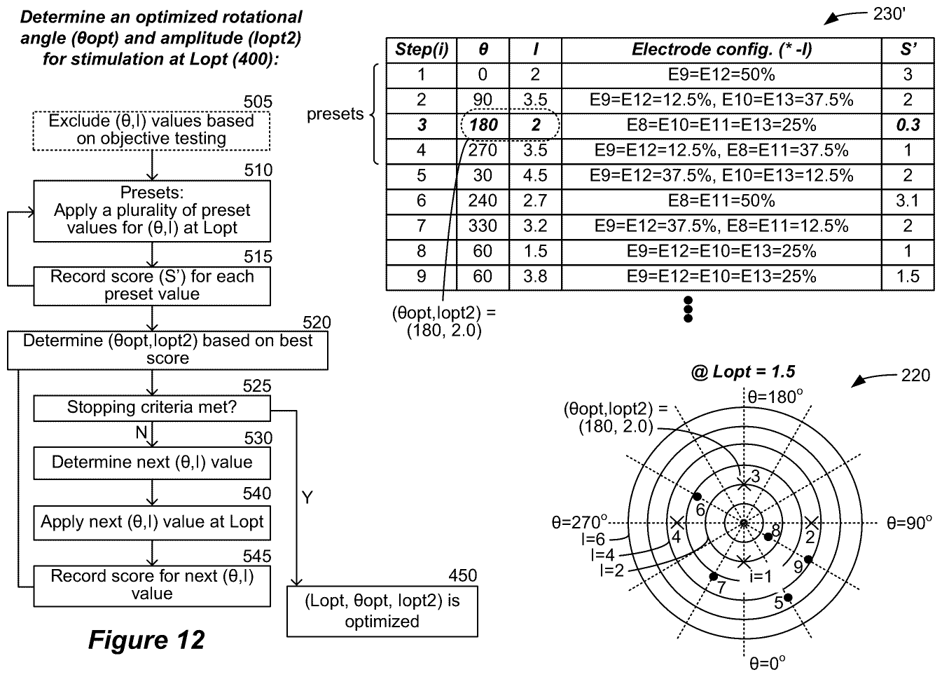

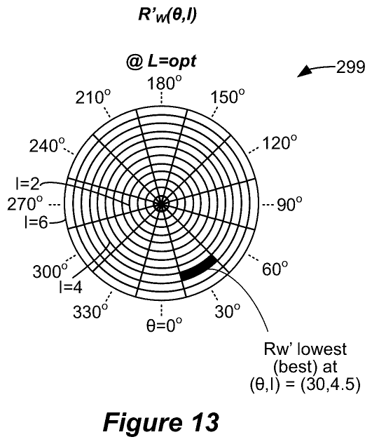

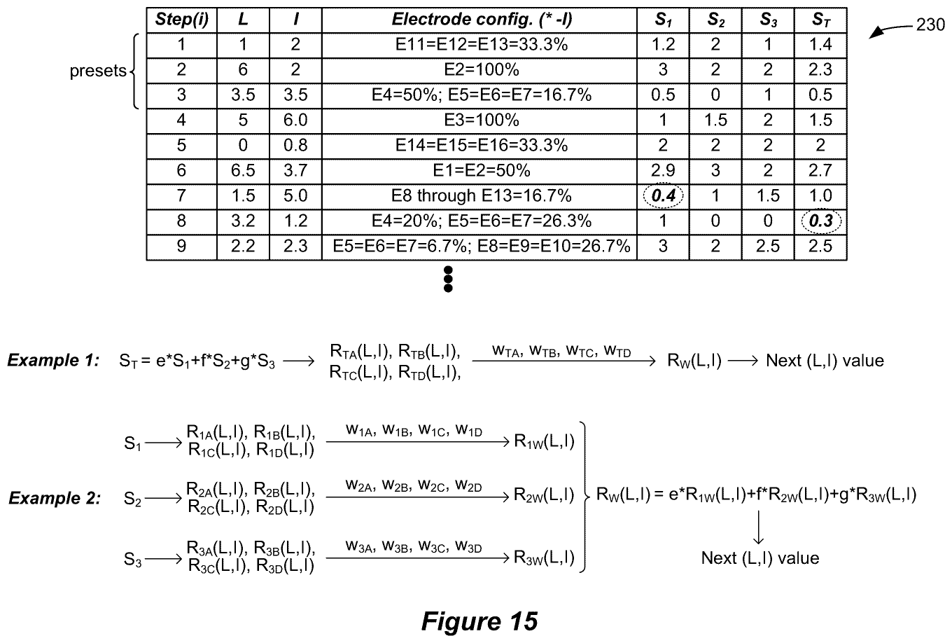

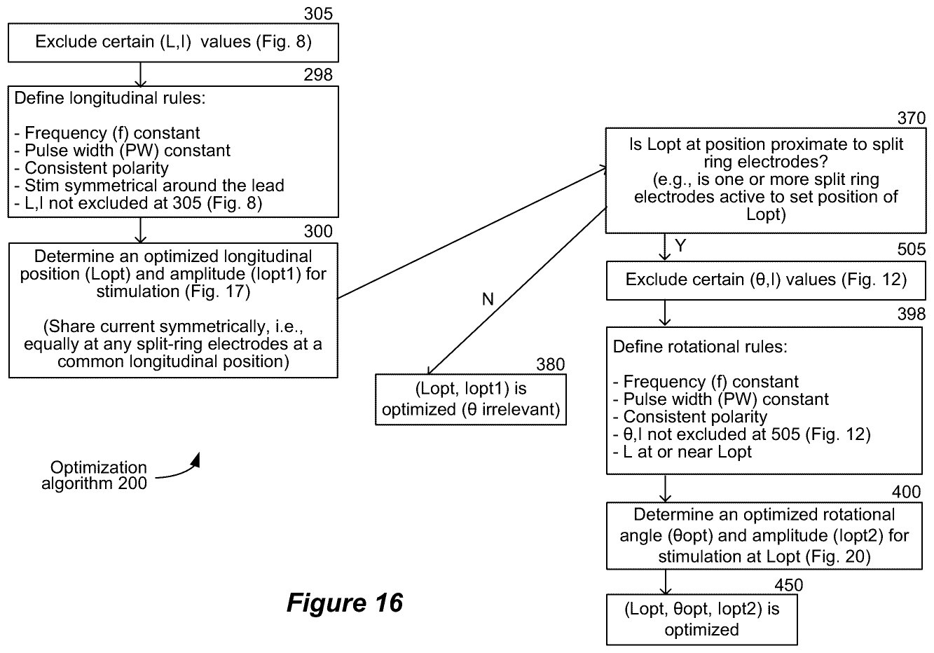

Note that there may be more than one best value: for example, two (L,I) values may have the same lowest score. In this case, and although not illustrated, the algorithm 200 may employ tie-breaking rules at step 320 to select a single optimal (L,I) value. For example, from amongst the various potential (L,I) values that are tied, the (L,I) value with the lowest amplitude I, or the lowest energy consumption, may be selected. If more than one score is made at each of the tested values, a point discussed further below with respect to FIG. 15, a value with a best average score, or a best single score, may be selected. It may also be preferred or not preferred to select a (L,I) at a particular longitudinal location. For example, it may be preferred to select as optimal an (L,I) value at a longitudinal value that is proximate to split ring electrodes, as this may allow the algorithm 200 to further optimize the rotational angle of the stimulation, as discussed further below with reference to FIGS. 12 and 13. Conversely, it may be preferred to select as optimal an (L,I) value at a longitudinal value that is not proximate to split ring electrodes; this may simplify optimization because rotational angle does not need to be optimized at such longitudinal positions. Other factors may also be used to break a tic between the scores of (L,I) values reflected in data set 230. For example, the optimal (L,I) may be selected as that having the least side effects, or based on other factors of convenience or efficacy.

Note that (Lopt, Iopt1), while optimized for the patient in the manner explained above, is not necessarily the best (L,I) value for the patient: some other (L,I) value not suggested as a next value by the algorithm 200, and therefore not tested, might actually correspond to a best value (e.g., lowest score S). Nevertheless, (Lopt, Iopt1) can still be said to be optimized for the patient, because the algorithm 200 searches the L,I parameter space 210 efficiently to arrive at a best value of (Lopt, Iopt1) for the patient.

At this point, the optimization algorithm 200 can determine whether the rotational angle θ at which stimulation will be applied (Lopt) should also be optimized. This depends on the determined position of Lopt, and in particular whether Lopt is proximate to split ring electrodes (370), which can require the algorithm 200 to consider the shape and placement of the electrodes on the lead. Referring again to FIG. 7, notice that if Lopt≥4.0, Lopt is not proximate to any split ring (directional) electrodes on lead 19, and there is therefore no reason to optimize rotational angle. In this circumstance, optimization by the algorithm 200 is complete, with (Lopt, Iopt1) determined as optimal for the patient (380). In other words, an optimal stimulation parameter set has now been determined for the patient: as explained above, from Lopt, the algorithm can determine (using the electrode configuration algorithm) the active electrodes, their polarities, and the percentage of Iopt1 that each active electrode should receive, which along with other non-optimized parameters (e.g., F, PW) comprises the optimized stimulation parameter set.

By contrast, if Lopt<4.0, then split ring electrodes are proximate to Lopt in this example. Note that whether Lopt is proximate to split ring electrodes can depend on the electrode configuration used to set Lopt at that longitudinal position, and whether that electrode configuration involves the use of split ring electrodes. For example, and referring to data set 230, it is seen that split ring electrodes E8-E13 are involved in setting (Lopt, Iopt1)=(1.5, 5.0), which allows the algorithm 200 to conclude that Lopt is proximate to split ring electrodes (370), because at least one split ring electrode is active to fix the position of Lopt.

When the algorithm 200 determines that Lopt is proximate to split ring electrodes (370) as in the depicted example, the algorithm 200 can proceed to determine an optimal rotational angle θopt for the application of stimulation at this longitudinal position Lopt (400), as shown in further detail in FIG. 12. Similar to the manner in which longitudinal position was simultaneously optimized (Lopt) with amplitude (Iopt1), the rotational angle of stimulation is also preferably simultaneously optimized (θopt) with amplitude (Iopt2). Because changing the rotational angle θ of the stimulation around the lead 19 changes the tissue receiving the stimulation, it is likely that the amplitude optimized at θopt will be different (Iopt2) from that determined when non-directional stimulation was provided (Iopt1).

Optimizing rotational angle θ in algorithm 200 involves trying different angles θ and amplitudes I at Lopt until θopt and Iopt2 are determined. As before, this process is iterative, and involves analogous steps (510-545) as occurred during longitudinal optimization of the stimulation (FIGS. 8-11F), as explained in the above-incorporated ‘950 Publication. Optimizing rotational angle θ involves the use of θ,I parameter space 220 and data set 230’ as shown in FIG. 12.

As before, in optional step 505, certain (θ,I) values may be excluded from further consideration and testing in the algorithm 200. Such (θ,I) values may be excluded based on the factors discussed earlier in analogous step 305, such as expected ineffectiveness (e.g., as based on preliminary testing), based on objective measurements (ERNAs), upon consideration of the tissue imaging information 114, etc., although it is only necessary to consider such factors at (or near) the optimal longitudinal position (Lopt) already determined.

Referring again to FIG. 12, preset values (θ,I) (510) are chosen to cover a considerable or relevant portion of the possible (θ,I) values at Lopt not excluded in θ,I parameter space 220. In the example shown, four presets are used with (θ,I) values of (0°, 2 mA), (90°, 3.5 mA), (180°, 2 mA), and (270°, 3.5 mA). Again, these are just example presets, and different values could be chosen, and different numbers of presets used. Note that the current amplitude of the presets can also be selected in light of Iopt1 as determined earlier during longitudinal optimization (FIG. 8). For example, because Iopt1=5.0 mA was determined earlier, current amplitudes equal to or closer to this value may be chosen for the (θ,I) presets. For example, presets of (0°, 4 mA), (90°, 5.5 mA), (180°, 4 mA), and (270°, 5.5 mA) may be used.

Data set 230′ keeps track of these values, and also stores (again with help of the electrode configuration algorithm) the electrode configurations needed to provide the stimulation at these different rotational locations. Data set 230′ may be a continuation of the data set 230 used during longitudinal optimization (FIG. 8), or it may comprise a separate data set. Similar to what occurred during longitudinal optimization, the algorithm will determine a next (θ,I) value to test using scores taken at the four presets. These details are explained in the '950 Publication, and because they are similar to steps summarized earlier during longitudinal optimization, they are only quickly summarized here.

As before, the preset (θ,I) values are applied to the patient at Lopt (510). This causes the clinician programmer 70 to transmit stimulation parameter sets indicative of θ and Lopt (as reflected in the electrode configuration) and amplitude I to the IPG 10 so that stimulation can be produced at the prescribed angle and longitudinal position. As each of these presets (θ,I) is sequentially applied to the patient (510), at least one score S′ at each of the presets is determined (515) and entered into data set 230′ using the GUI 99. Such scores as before may be subjective or objective. Note in the description of rotational optimization that follows that variables are given a prime symbol (e.g., S′, R′, W′) to differentiate them from variables used earlier (FIGS. 8-11F) during longitudinal optimization.

After sequentially applying stimulation according to these presets and determining and recording their scores S′ after patient testing, the programming algorithm 200 can determine a best of the (θ,I) values (θopt, Iopt2) tested to this point based on the scores S′ provided at each of the previously-tested positions (520). As the algorithm 200 iterates, more (θ,I) values will be tested and scored, and (θopt, Iopt2) can be updated accordingly at this step. At this point, after only testing the presets, (θopt, Iopt2) is determined at step 520 to be (180°, 2) (at step i=3), because this tested value yields the best (e.g., lowest) score (S′=0.3).

Next, the algorithm 200 determines whether one or more stopping criteria has been met (525), and stopping criteria can be similar to those described earlier for longitudinal optimization. If not, the algorithm 200 continues to determine a next (θ,I) value to be tested (530) in a next iteration of the algorithm (step i=5).

These details at step 530 mimic the sub-steps shown earlier in FIG. 10 during longitudinal optimization, but are not redundantly illustrated here for rotational optimization. As before, the algorithm 200 computes and considers one or more factors, such as R′_{A}, R′_{B}, R′_{C}, R′_{D}, although again more or fewer factors could be used. Each of these factors is preferably determined at all possible (θ,I) points in the θ,I parameter space 220, although certain (θ,I) values can also be excluded, as explained earlier.

Factors R′_{A}, R′_{B}, R′_{C}, and R′_{D} largely mimic their longitudinal counterparts R_{A}, R_{B}, R_{C}, and R_{D}, and comprise determinations of inverse distance (R′_{A}), absolute distance (R′_{B}), distance variance (R′_{C}), and a lower amplitude preference (R′_{D}). These factors and their resulting data sets as used during rotational optimization are not shown in the Figures, but are explained in the above-incorporated '950 Publication, including descriptions how the calculations of these factors can be varied in a rotational environment.

As before, a weighted factor data set 299 R′_{W} for all (θ,I) positions is determined using the factors R′_{A}, R′_{B}, R′_{C}, R′_{D}, determined earlier, which can involve the use of weights W′_{A}, W′_{B}, W′_{C}, w′_{D}. (R′_{W}(θ,I)=W′_{A}*R′_{A}(θ,I)+W′_{B}*R′_{B}(θ,I)+W′_{C}*R′_{C}(θ,I)+W′_{D}*R′_{D}(θ,I)). Once R′_{W} is determined at each of the (θ,I) values (perhaps with some values excluded), a best R′_{W}(θ,I) value is selected from data set 299, which determines the next (θ,I) value to be tested (530). In the example shown in FIG. 13, the best (e.g., lowest) R′_{W}(θ,I) value is assumed to occur at value (30°, 4.5), which then comprises the next (θ,I) value to be tested at Lopt (during step i=5). As before, if there is potentially more than one best value R′_{W} in data set 299, the algorithm 200 can employ tie-breaking rules.

Referring again to FIG. 12, once the next (θ,I) value has been determined (530), that value (30°, 4.5 mA) can be populated into data set 230′ as a next step (i=5) and thus as a next point to be tested by the clinician. The electrode configuration algorithm can determine an electrode configuration suitable to position stimulation at this new rotational position and at Lopt, as shown in data set 230′. Once the electrode configuration is determined, this next (θ,I) value (the stimulation parameter set) is transmitted and applied to the patient (540) similarly to what was described earlier for the preset (θ,I) values. At least one score (S′=2) is then determined for stimulation occurring at this new (θ,I) value and is recorded into the data set 230′ (545) using the GUI 99.

At this point, the algorithm 200 can return to step 520, wherein a best of the tested values (θopt, Iopt2) is determined and/or updated. (θopt, Iopt2) will remain as (180°, 2.0) (step i=3), because this step shows the best (e.g., lowest) score S′ to this point. Assuming a stopping criterium isn't met (525), the algorithm 200 continues iterating and determines a next (θ,I) value to be tested (530) (step i=6), which is applied 540 and for which a score S′ is recorded 545, etc. The effect of such successive iterations of the algorithm 200 is shown in the data set 230′ and the θ,I parameter space 220 of FIG. 12, which shows the (θ,I) values that are determined and how they logically and efficiently traverse the θ,I parameter space 220 as dictated by factors R′_{A}-R′_{D} and their weightings. Again, the data shown in FIG. 12 is fictitious and only provided to help illustrate operation of the algorithm 200.

If a stopping criterium has been met (525), no further (θ,I) values are determined or tested, and an optimal value of (θ,I)—(θopt, Iopt2)—is determined, which would comprise the (θ,I) value determined and updated earlier during step 520. In the illustrated example, (θopt, Iopt2) corresponds to the lowest score S′ (0.3) when θopt=180° and Iopt2=2.0 mA (step i=3). Notice in this example that (θopt, Iopt2) happens to correspond with one of the presets, but this is coincidental and wouldn't necessarily occur.

At this point, stimulation for the patient has been optimized (450), with Lopt optimized during longitudinal searching, and (if necessary, and depending on Lopt's proximity to split ring electrodes) with θopt and Iopt2 optimized during rotational searching at Lopt. To summarize, optimization algorithm 200 has determined an optimized stimulation parameter set (Lopt, θopt, Iopt2)=(1.5, 180°, 2.0 mA) for the patient.

Note that Lopt and θopt, pursuant to the electrode configuration, defines how this amplitude Iopt2=2 mA should be split between the electrodes. As shown in the data set 230′ (step i=3), the current Iopt2 should be split equally between electrodes E8, E10, E11, and E13, with each of these electrodes receiving −0.5 mA, which will place the stimulation at the optimal longitudinal (Lopt=1.5) and rotational (θopt=180°) positions relative to the lead 19. Again, other stimulation parameters—such as frequency F and pulse width PW are included as part of the optimized stimulation parameter set, which are assumed to have been optimally determined elsewhere. Like amplitude I, these other stimulation parameters could be optimized using the disclosed technique as well. As was the case with longitudinal optimization, the algorithm 200 may apply tie-breaking rules to select an optimal (θopt, Iopt2) value from between otherwise equally-valued scores S′ at step 520. Again, note that (θopt, Iopt2), while optimized for the patient in the manner explained above, is not necessarily the best (θ,I) value for the patient: some other (θ,I) value at Lopt not suggested as a next value by the algorithm 200, and therefore not tested, might actually correspond to a best value (e.g., lowest score S′) for the patient. Nevertheless, (θopt, Iopt2) can still be said to be optimized for the patient, because the algorithm 200 still searches the θ,I parameter space 220 efficiently to arrive at a best value of (θopt, Iopt2) for the patient at Lopt. In this sense, (θopt, Iopt2) can be said to be optimized, or comprise an optimal value, for the patient.

FIG. 14 shows the GUI 99 that may be used to implement the optimization algorithm 200, and displays information that may be useful to the clinician as the algorithm 200 operates. GUI 99 may include a user-selectable option allowing the clinician to “run programming algorithm 200.” Upon selection of this option, a longitudinal optimization section 550 may be displayed, including aspects of data set 230 and parameter space 210 that are useful in understanding how the algorithm 200 is progressing during longitudinal optimization. As noted earlier, data set 230 displays preset and next-determined values (L,I), and allows the clinician to input the score (S) at each. Once a stopping criterium is met, the GUI 99 can display the determined values for Lopt and Iopt1, along with other useful information such as its (best) score S, and perhaps even the electrode configuration and/or stimulation parameters that places the stimulation at that position.

If the algorithm 200 determines based on Lopt that rotational optimization is recommended (see FIG. 8, 370), this fact can be displayed to the clinician as a selectable option (552) to allow such rotational optimization to commence. Otherwise, the GUI 99 can display that rotational optimization is not necessary, and that optimization is (Lopt, Iopt1) is complete (not shown). If rotational optimization is undertaken, the GUI 99 can display a rotational optimization section 555, including aspects of data set 230′ and parameter space 220 that are useful in understanding how the algorithm 200 is progressing during rotational optimization. As before, data set 230′ displays preset and next-determined values (θ,I), and allows the clinician to input the scores (S′) at each. Once a stopping criterium is met, the GUI 99 can display the determined values for Lopt, θopt, Iopt2, along with other useful information such as its (best) score S′, and perhaps even the electrode configuration and/or stimulation parameters that places the stimulation at that position. Although not shown, the GUI 99 of FIG. 14 may also include a picture of the lead for which stimulation is being optimized, along with the location of the cathode pole 120 that is formed by the optimized stimulation, similar to what was shown in FIG. 5A. The GUI 99 may also display the tissue imaging information 114, which as noted earlier may be important to review at various portions of the algorithm 200.

Notice that programming algorithm 200 addresses problems of determining optimal stimulation for DBS patients. As mentioned earlier, in a typical DBS system there are many combinations of I, L, and θ that that can be tested and scored when determining optimal stimulation parameters, and testing all such combinations is burdensome and impractical during a programming session. Use of the programming algorithm 200 efficiently and automatically selects next values to test, and can automatically decide when enough values have been tested. As such, much of the guess work in selecting optimal stimulation parameters is removed, and optimal stimulation parameters can be arrived at efficiently and in a reasonable period of time, such as during a typical programming session.

Many modifications can be made to the programming algorithm 200 as described up to this point. Use of the algorithm 200 has been described as particularly useful when used to determine stimulation parameters for a patient having a directional lead (e.g., 19) with split ring electrodes at least some longitudinal positions on the lead. With such a lead, both longitudinal optimization and rotational optimization can be useful. However, algorithm 200 may also be used in part to provide only longitudinal optimization or only rotational optimization. For example, only longitudinal optimization aspects of the technique (e.g., FIGS. 8-11F) may be used to determine Lopt in a directional lead 19; rotational optimization (FIGS. 12 and 13) may not be necessary, or if necessary could occur via other means. Likewise, only rotational optimization aspects of the technique may be used to determine θopt at a particular longitudinal position along a directional lead, which may be particularly useful if the longitudinal position of the stimulation along the lead (e.g., Lopt) is already known or has been determined by other means. Even if Lopt is not known, the algorithm 200 may still apply only rotational optimization at a given longitudinal position L of the clinician's choosing.

Furthermore, while the algorithm 200 has been described sequentially as comprising longitudinal optimization followed by rotational optimization, the order could be reversed. Still further, longitudinal optimization and rotational optimization can occur more than once. For example, longitudinal optimization may occur to determine Lopt1; followed by rotational optimization to determine θopt1 at Lopt1; followed by further longitudinal optimization to perhaps further optimize Lopt2 at θopt1; followed by further rotational optimization to perhaps further optimize θopt2 at Lopt2; etc.

The algorithm 200 may also be used with non-directional leads (e.g., 18, FIG. 1B) having only circumferential ring electrodes, or with paddle leads. See, e.g., U.S. Pat. No. 10,149,979 (describing paddle leads in a Spinal Cord Stimulation (SCS) system). In these circumstances, only longitudinal optimization aspects of the technique may be necessary.

FIG. 15 shows another modification to programming algorithm 200. In this modification, the clinician can enter a plurality of scores S at each tested stimulation parameter at each iteration of the algorithm. FIG. 15 illustrates this only with respect to longitudinal optimization for simplicity, although this could also be applied to rotational optimization as well. As shown, three scores S₁, S₂, and S₃ are determined at each step, although two or more than three could also be considered. For example, Si can comprise a bradykinesia score; S₂ a rigidity score; S₃ may comprise a score determined based on objective testing, such as the ERNA measurements described earlier. Again, these are just examples; other subjectively determined or objectively measured patient outcomes can be scored as noted earlier. See U.S. Patent Application Publication 2021/0196956 (discussing determination of a plurality of scores for a given stimulation parameter set in a DBS application).

In Example 1, the scores S₁, S₂, and S₃ are weighted (per weights e, f, and g) to arrive at S_{T}, which is then used to assist in selecting a next value to be tested. Note that such weighting can comprise averaging the scores S₁, S₂, and S₃. Once S_{T} values are determined at previously tested locations, the algorithm 200 can proceed as before to determine a next value to be tested. Thus, and in light of scores S_{T} determined at earlier steps, data sets R_{TA}(L,I) to R_{TD}(L,I) can be determined, and these can be weighted to determine a weighted data set R_{W}(L,I), which can be used to choose the next value ((L,I) in this case) to be tested by determining a best (e.g., lowest) value in R_{W}(L,I).

Example 2 also involves determining a weighted data set R_{W}(L,I) that can be used to select next values to be tested, although this doesn't use S_{T} to do so. Instead, in this example, each of the individual scores S₁, S₂, and S₃ are processed separately to arrive at a weighted data set associated with that score (R_{1W}(L,I), R_{2W}(L,I), and R_{3W}(L,I)), and then these weighted data sets are weighted again (per e, f, and g) to arrive at data set R_{W}. This example is beneficial because the weights used to form the R_{iW}(L,I) data set for each score Si can be set differently if desired (e.g., W_{iA} to W_{iD}). Again, use of multiple scores S′ can also be used during rotational optimization to determine a next (θ,I) value to be tested.

Regardless of how R_{W}(L,I) is determined and used to select next points to be tested, the algorithm 200 can use any of the scores to ultimately select optimal stimulation parameters (in this case, Lopt and Iopt1). For example, once data set 230 is complete and all values have been tested, ST can be assessed to determine Lopt and Iopt1 in this example, which may make sense as ST comprises a general averaging of the individual scores S₁, S₂, and S₃. If S_{T} is used in this manner, the algorithm 200 would determine that Lopt=3.2, and Iopt1=1.2 mA (step i=8), because this step corresponds to the best (lowest) value for S_{T} (0.3) in data set 230. Alternatively, the algorithm 200 could use any of the individual scores in data set 230 to determine optimal parameters. Assume for example that S₁ scores a particularly important symptom such as bradykinesia. The algorithm 200 may thus use this score S₁ to determine the optimal parameters. If S₁ is used in this manner, the algorithm 200 would determine that Lopt=1.5, and Iopt1=5.0 mA (step i=7), because this step corresponds to the best (lowest) value for S₁ (0.4) in data set 230. S_{T} by contrast may be used only to assist in selecting the next values to be tested, or may not be used at all. Still alternatively, algorithm 200 could assess all scores S₁, S₂, S₃, and S_{T}, and use the best (lowest) value of all of these to select the optimal parameters.

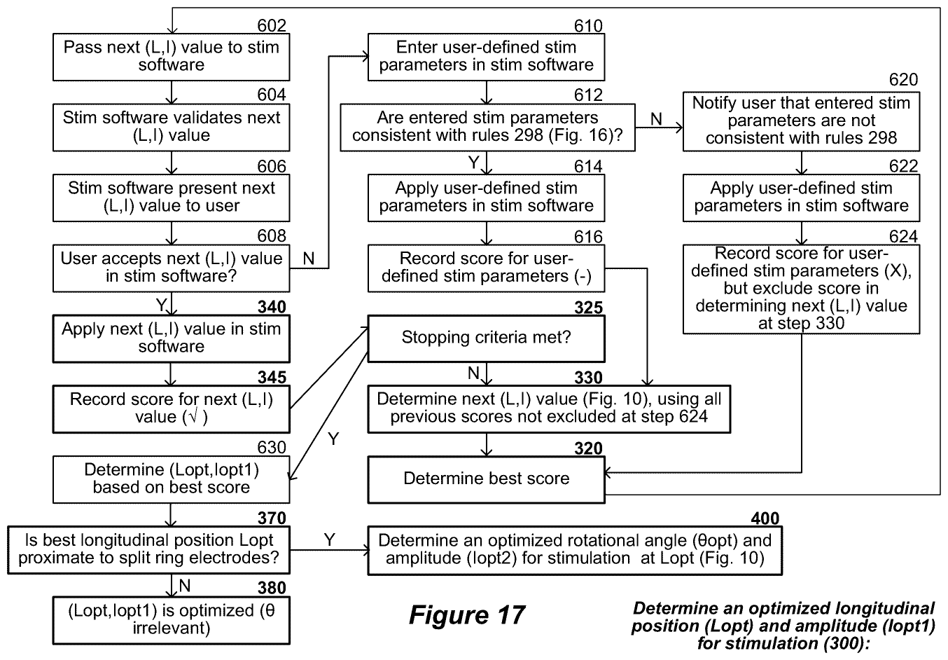

To this point, optimization algorithm 200 has been illustrated as operating in an iterative manner, where suggested next (L,I) and (θ,I) values in L,I and θ,I parameters spaces 210 and 220 are tested and scored. However, the algorithm 200 can also be modified as shown in FIGS. 16-20 to allow a user more flexibility to test various (L,I) and (θ,I) values, or other user-defined stimulation parameters more generally, even if those values are not suggested by the algorithm. This adds desired flexibility to the algorithm 200, because it provides clinician the ability to test other user-defined stimulation parameters beyond those suggested by the algorithm. In certain instances, scores determined for the patient using such user-defined stimulation parameters may be used by the algorithm as it iterates, for example to predict next (L,I) and (θ,I) values. Alternatively, the algorithm 200 may simply record such scores without using them to predict next (L,I) and (θ,I) values.

FIG. 16 shows this modification to algorithm 200 at a high level, which is in certain respects similar to FIG. 6, and uses similar element numbers. Newly added are steps 298 and 398 which prescribe rules to be used during longitudinal and rotational optimization respectively. As described in detail later, user-defined stimulation parameters may be handled differently depending on whether such parameters are consistent with rules 298 or 398.

Rules specified at steps 298 and 398 are designed, generally speaking, to allow the algorithm 200 to determine whether user-defined stimulation parameters entered by the user vary significantly with respect to assumptions that the algorithm 200 uses when determining next (L,I) or (θ,I) values to suggest and test. For example, because the algorithm 200 seeks to optimize amplitude I and stimulation position (both longitudinally (L) and possible rotationally (0) around the lead), it may be desirable for the algorithm 200 to assume that other stimulation parameters provided during testing, such as the pulse width (PW) and frequency (F) of the pulses, are held constant. This is reasonable, because allowing changes to pulse width and frequency would affect the energy delivered to the patient, making optimization of amplitude (which also affects energy) difficult, thus upsetting predictive assumptions underlying which (L,I) and (θ,I) value to test next. Thus, while changing the pulse width and/or frequency of the stimulation used during operation of the algorithm 200 is permitted to enhance user flexibility, as shown below, results obtained during such changes may or may not be used to affect how the algorithm iterates when determining next (L,I) or (θ,I) values to test.

Other rules at steps 298 and 398 are designed to assess whether the nature of the stimulation used during testing is too variable in other respects. For example, rules 298 may assess whether the polarity of the stimulation is significantly varied. For example, and discussed earlier, stimulation in a DBS application is typically provided using monopolar stimulation, with stimulation provided at one lead-based cathodic pole (120) and using the case electrode Ec as an anodic return (120, FIG. 5A) (cathodic monopolar stimulation). While the algorithm 200 includes the flexibility to allow a user to test a different polarity—e.g., by providing an anodic pole and using the case as a cathodic return (anodic monopolar stimulation), or by providing bipolar or multipolar stimulation at the lead (multipolar stimulation)—this may be too radical of a change for the algorithm 200 to consider when predicting next (L,I) and (θ,I) values to test. Rules 298 and 398 may thus define a consistent polarity—such as cathodic monopolar stimulation—to be used by the algorithm 200 as it iterates and suggests next values to test.

Rules 298 may allow the algorithm 200 to assess other relevant considerations. For example, during longitudinal optimization, the algorithm 200 may prefer when predicting a next (L,I) to not consider user-defined stimulation parameters that provide stimulation that is not symmetric around the lead—i.e., to not consider stimulation that is not shared equally among the electrodes at the longitudinal position being tested. Rules 298 may also prefer to not consider user-defined simulation parameters that were excluded because they are outside the relevant L,I parameter space 210 being tested. Such exclusions can be defined by the user at an optional step 305, which was described earlier (see FIG. 8). If user-defined stimulation parameters comprise values for L or I outside of this space 210, results (scores) obtain when testing such stimulation may not be used when predicting next (L,I) values to test.

The rules prescribed at step 398 precede rotational optimization, once longitudinal optimize (Lopt) has been completed. Again, rotational rules 398 may require that user-defined stimulation parameters are at a constant frequency and pulse width, and that a consistent polarity is used. Rules 398 may also prescribe use of values for θ and I that were not otherwise excluded from the relevant θ,I parameter space 220 being tested at optional step 505 (see FIG. 12). Rules 398 may also prescribe that user-defined stimulation parameters must be at or near Lopt determined earlier. Such rules for longitudinal positioning during rotational optimization may depend on the nature of the scores (S′) being considered. For example, if score S′ only quantify efficacy, rules 398 may specify that stimulation must be provided at exactly at Lopt. If score S′ quantifies side effects, at least in part, rules 398 may specify that stimulation can be provided at a limited range around Lopt (e.g., Lopt−Δ<L<Lopt+Δ).

The rules 298 and 398 upon which the algorithm 200 will iterate are preferably user definable. For example, the user will have likely determined a pulse width, frequency, and a consistent polarity (e.g., cathodic monopolar stimulation) to use (at least primarily) during testing. Such values may be input to the GUI 99 prior to the start of the algorithm 200, such as by using the interfaces 104 and 105 described earlier (FIG. 5A). The algorithm 200 can also include default rules 298 or 398 defining how testing will proceed, which the user can then accept, modify, or exclude (using GUI 99). Rules 298 or 398 may include more or less rules beyond those illustrated here, and exclusive lists are not shown.

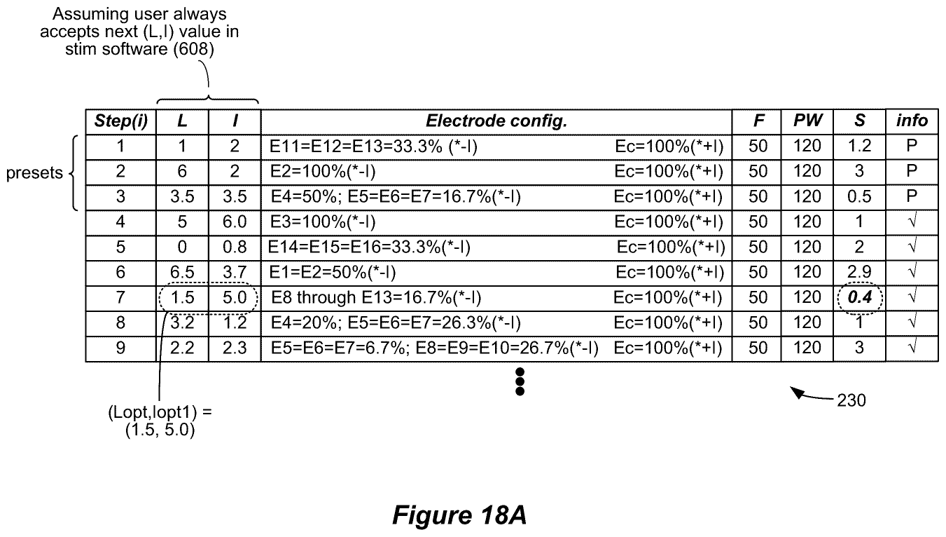

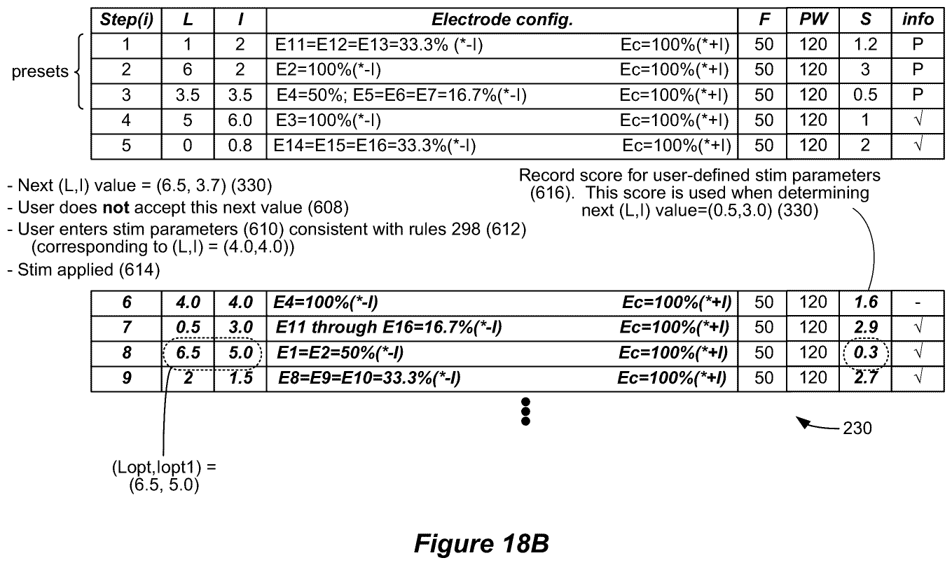

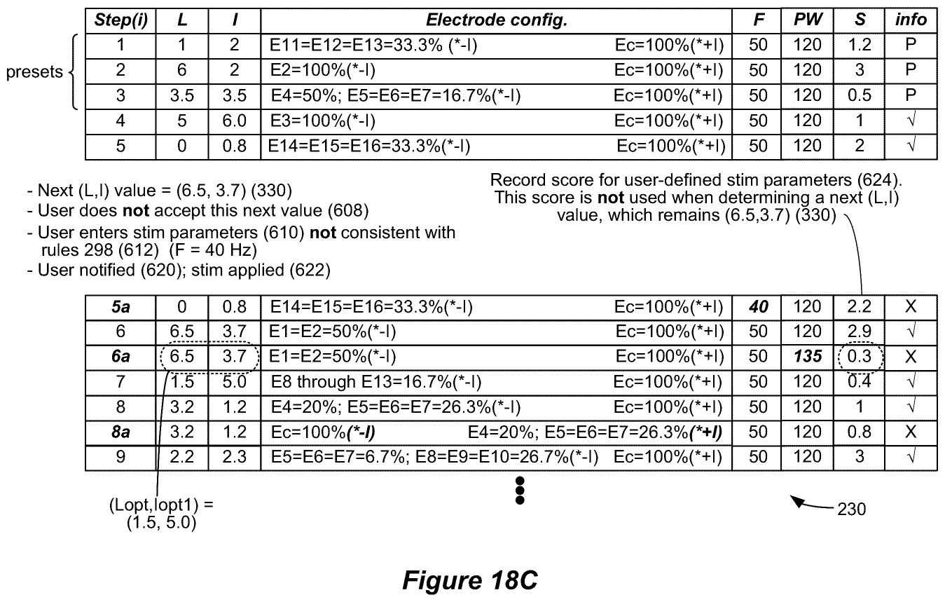

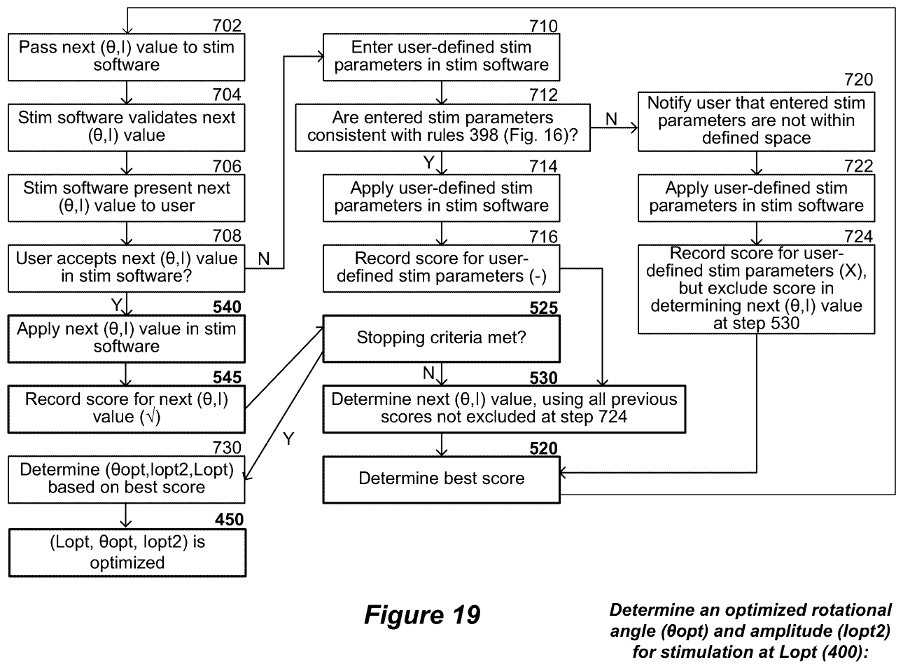

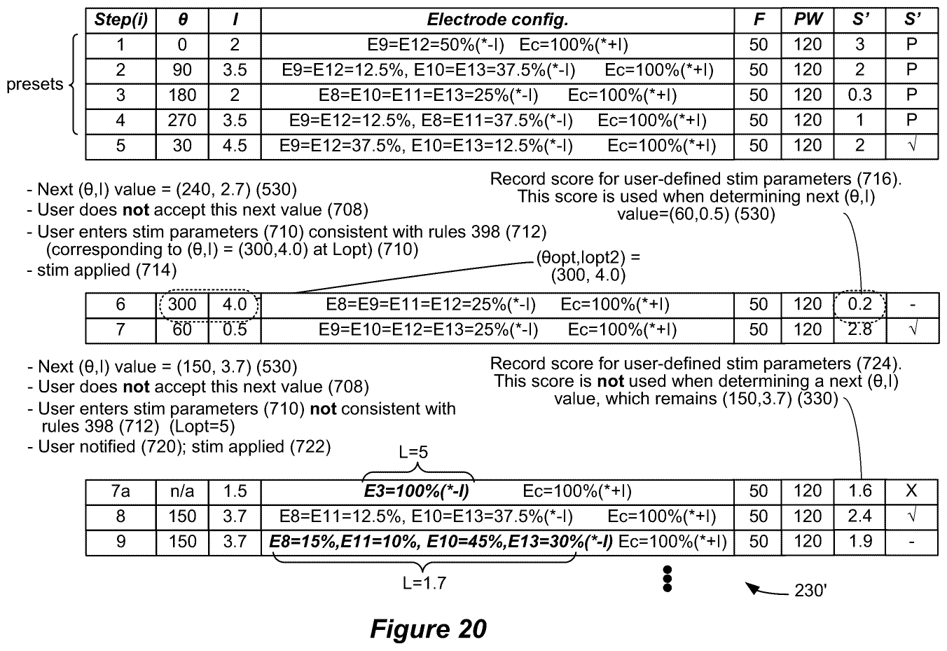

FIGS. 17-18C describe operation of the modified algorithm 200 of FIG. 16 used during longitudinal optimization (determining Lopt1, Iopt1) (300), while FIGS. 19 and 20 describe operation of the modified algorithm used during rotational optimization (determining θopt, Iopt2 at Lopt) (400).

FIG. 17 shows the flow used during longitudinal optimization, and is somewhat similar to the longitudinal flow 300 described earlier (FIG. 8), and shows the steps of that earlier flow in bold so that newer aspects can be appreciated. The flow of FIG. 17 describes iterative operation of the algorithm 200, and thus for simplicity does not illustrate initial steps, such as testing preset values (L,I) as described earlier.

As described earlier, at some point, the algorithm 200 will have predicted a next (L,I) values to test (330), and (skipping step 320) this next (L,I) value is passed to the stimulation software (602). The stimulation software, like the algorithm 200, may comprise part of the external system software 96 that generates the GUIs (99, FIGS. 5A, 14) and otherwise allows a user to program the IPG 10. While the stimulation software is described here as being accessible by the algorithm 200, it may also comprise part of the algorithm 200 itself. If necessary the stimulation software can verify this next (L,I) value (604), i.e. to verify that the IPG can indeed be programmed to produce stimulation using these parameters, and that such parameters are otherwise compliant with the simulation software. In this regard, the stimulation software may apply certain rules, such as only allowing the use of stimulation parameters that would be safe for the patient.

Once verified, this next (L,I) value can be presented to the user (606), e.g., as part of data set 230 as depicted in the GUI 99 of FIG. 14. At step 608, the user can choose to accept this next (L,I) value or not. This detail isn't shown in FIG. 14 for simplicity. If the user chooses to accept this next suggested (L,I) value, it is tested as described before: the next (L,I) value is applied at the IPG 10 (using the stimulation software) (340), and a score is determined and recorded in data set 230 for that value (345). (As described earlier, one or more score may be determined; see FIG. 15). The algorithm 200 then checks to see if a stopping criterium has been met (325), as described earlier. If not, a next (L,I) value is determined as described earlier (sec FIG. 10) (330), using earlier-tested (L,I) values. However, certain earlier-tested user-defined stimulation parameters may not be considered at this step, as discussed later with respect at step 624.

If one assumes that the user always accepts the next (L,I) values to test as suggested by the algorithm 200, the data set 230 will iteratively populate as shown in FIG. 18A. FIG. 18A thus shows the same data set 230 that was described earlier during longitudinal optimization (see FIG. 8), although additional details are shown so that the rules of step 298 can be better appreciated. For example, notice that the electrode configuration more fully describes the cathodic (−) and anodic (+) pole represented, showing in this case use of a consistent polarity (cathodic monopolar stimulation) at all values tested. Also, the pulse width (PW) and frequency (F) of the stimulation provided are shown, and are constant at all tested values, again as required by rules 298. As before, an optimal (L,I) value of Lopt, Iopt1=1.5, 5.0 has been determined by the algorithm in FIG. 18A, because this setting produced the best (e.g., lowest) score (630, FIG. 17). An informational column (info) is also provided to define the nature of the stimulation parameters tested at each iteration. “P” indicate preset values, while entries denoted with a check mark (✓) denote use of a next (L,I) value suggested by the algorithm 200. These information indicators can also comprise different flags associated with the data in data set 230.

Returning to FIG. 17, and as discussed earlier, a user may not always wish to accept a next (L,I) value suggested by the algorithm 200 to test (608), and instead the user may wish to test other user-defined stimulation parameters. These user-defined stimulation parameters may or may not be consistent with rules 298, and thus treated differently by the algorithm. When a user does not accept a next suggested (L,I) to test at (608), and instead enters different user-defined parameters at (610) in the GUI 99, the algorithm 200 assesses whether these user-defined stimulation parameters are consistent with rules 298 (612).

FIG. 18B shows an example where entered user-defined stimulation parameters are consistent with rules 298. This example shows the data set 230 as it has populated to step i=5. At this point, the algorithm 200 has determined a next (L,I) value=6.5,3.7) to test at next iteration i=6, but the user does not accept this (608), and enters different user-defined stimulation parameters. However, these stimulation parameters are still consistent with rules 298, even though not suggested by the algorithm: they comprise a pulse width of 120 μs and frequency of 50 Hz consistent with the constant values established by rules 298 (and as used at all other steps). These simulation parameters also use cathodic monopolar stimulation, consistent with the polarity established by rules 298. The stimulation is also symmetrical around the lead, and not positioned at excluded L or I values, again as prescribed by rules 298. In effect, the user-defined stimulation parameters are consistent with an L,I value of corresponding to (4.0, 4.0). In this regard, note that the user could at step 610 enter the user-defined stimulation parameters as an L,I value, or could enter “raw” stimulation parameters (e.g., an electrode configuration and an amplitude) from which a corresponding L,I value can be determined.

At this point, the stimulation software can apply these user defined stimulation parameters (at (L,I)=(4.0, 4.0)) (614), and a score can be recorded for this stimulation (S=1.6) (616), as shown at step i=6 in the data set 230. The information column may use a different indication or flag to denote this iteration of the algorithm as one in which user-defined stimulation parameters were entered that were consistent with rules 298, as denoted by a dash (−).

Returning to FIG. 17, notice at this point that the algorithm 300 would normally determine whether a stopping criterium defined for the algorithm 200 has been met (see steps 345, 325). Stopping criteria were explained earlier. However, when user-defined stimulation parameters are used, this step (325) can be skipped. This is preferable, because the stopping criteria may be set assuming that the algorithm 200 is predicting (and the user is using) next (L,I) values. Use of user-defined stimulation parameters may be in conflict with the assumptions upon which stopping the algorithm is based. For example, it is possible that user-defined stimulation parameters may be too close to a (L,I) value that was previously tested, therefore inadvertently or coincidentally trigging the algorithm to stop (325). For these reasons, it is preferable to bypass considering stopping criteria (325) immediately after user-defined stimulation parameters are used. (An exception may be if the score received for the tested user-defined simulation parameters is so good (e.g., 0) as to suggest that the algorithm 200 has identified Lopt, Iopt1 and should simply stop. While possible, this alternative isn't shown in FIG. 17 for simplicity).

With the score entered at this point (step i=6) (616), the algorithm 200 proceeds to determining a next (L,I) value to test (330) during a next iteration of the algorithm (i=7), as described earlier. Because the user-defined stimulation parameters at step i=6 were consistent with rules 298, results at this step (e.g., the score entered at i=6) can be used to predict the next (L,I) value to test. By way of review (FIGS. 10-11F), predicting next (L,I) values involves consideration of all previous (L,I) values tested as well as the entered scores at those values (assuming certain scores were not excluded at step 624, as discussed further below). In short, because the user-defined stimulation parameters entered at step i=6 are consistent with rules 298, the results from testing stimulation at this value can be used in the prediction of a next (L,I) value, even though these user-defined stimulation parameters themselves were not predicted by the algorithm 200.

In FIG. 18B, the next (L,I) value determined (330) is (0.5,3.0). Notice that the previously-determined next (L,I) value of (6.5,3.7), which was not accepted by the user (at i=6), has now been superseded, because the algorithm has considered the additional data point at i=6. After determining a best (lowest) score (320) to this point, the algorithm continues to iterate by validating and presenting the next (L,I) value to the user (602-606). If one assumes that the user in the example of FIG. 18B simply accepts next suggested (L,I) values to test from this point forward, the data set 230 would be populated as shown. Notice comparing FIGS. 18A and 18B that the next (L,I) values tested diverge at step i=6. This is because of the additional information the algorithm 200 received and considered by virtue of testing the rule-compliant user-defined stimulation parameters at step i=6.

In FIG. 18B, it is assumed that the algorithm 200 reaches a stopping criterium (325) after the (L,I) value at step i=9 is tested and scored. At this point, the algorithm 200 can determine Lopt, Iopt1 (630) based on the lowest score in the data set 230, which in this example corresponds to (6.5, 5.0).

FIG. 18C shows an example where user-defined stimulation parameters are not consistent with rules 298. This example shows the data set 230 as it has populated to step i=5. At this point, the algorithm 200 has determined a next (L,I) value=(6.5,3.7) to test at next iteration i=6 (330), but the user does not accept this value (608). Instead, the user enters user-defined stimulation parameters (610), specifying the use of a frequency of 40 Hz. It is assumed here that rules 298 require the frequency to be at a constant frequency of 50 Hz, as exemplified by the earlier iterations shown in data set 230. As such, the user defined stimulation parameters are not consistent with rules 298 (612). The GUI 99 may notify the user of this (620), and ask the user to confirm the input parameters for testing. At this point, the user-defined stimulation parameters are applied (622) and a score entered for them (S=2.2) (624), as shown in entry 5a in data set 230. Note that the other stimulation parameters (aside from frequency) are largely irrelevant, but in this example are shown for simplicity as corresponding with the parameters tested at i=5 (they could be different in other respects).

Because these stimulation parameters are not consistent with rules 298, entry 5a in data set 230, including its score, is not used by the algorithm 200 in determining next (L,I) values to test (at step 330). This is indicated in the information column by an X, which again may comprise a particular flag in the data set 230. Even though entry 5a will not be used to determine next (L,I) values, it is still important to record, in particular because this entry is still relevant to determining Lopt, Iopt1 (630). Because it is unnecessary to determine a next (L,I) value at this point, step 330 is skipped. The algorithm 200 also skips assessing whether a stopping criteria has been met (325) for the same reasons explained earlier in FIG. 18B-basically because it is not reasonable to stop based on the use of user-defined stimulation parameters as opposed to (L,I) values predicted by the algorithm 200.

Returning to FIG. 17, after determining the best (lowest) score to this point (320), the algorithm 200 returns to eventually present the next (L,I) value (606), which in this case had already been determined ((L,I)=(6.5,3.7)) after step i=5. This next (L,I) value was not initially accepted by the user (entry 5a), although it is assumed in FIG. 18C to now be accepted (608), and so is applied (340) and scored (345) at step i=6. Notice by comparing the entries at steps i=6in FIGS. 18A and 18C that the algorithm 200 essentially returns to where it was after handling and recording the non-rule-compliant entry at 5a. In this sense, the algorithm 200 does not diverge from its operation when handling such non-rule-compliant stimulation parameters.

Continuing the example of FIG. 18C, and assuming a stopping criteria isn't met (325), the algorithm 200 determines a next (L,I) value to test (330), which is only based on entries that were not flagged earlier at step 624 as being non-rule-compliant. Thus, and similarly to what shown in FIG. 18A, an (L,I) value of (1.5,5.0) has been determined using entries 1-6 (but not 5a).

In FIG. 18C it is assumed that the user again does not accept this next (L,I) value (608), and instead enters user-defined stimulation parameters (610) that are not compliant with rules 298 (612), this time because the pulse width (135 μs) has been varied (135 μs) from the constant value (120 μs) defined in rules 298. (As before, the other entered stimulation parameters are largely irrelevant). Nevertheless, these user defined stimulation parameters are eventually applied (622), and scored (624), as shown in entry 6a in the data set 230. Again, this entry 6a is flagged (X) and will not be used subsequently to determine next (L,I) values to test.

FIG. 18C also shows another example of non-rule-compliant user-defined stimulation parameters at entry 8a. Here, the frequency and pulse width are rule compliant, but the polarity of the stimulation is not consistent with cathodic monopolar stimulation used earlier and as specified by rules 298; instead, the user has specified the use of anodic monopolar stimulation. Again, these user defined stimulation parameters are applied (622), and scored (624), and flagged (X) as shown in entry 8a in the data set 230.

To summarize, FIG. 18C shows different examples of the use of user-defined simulation parameters that are not compliant with some (but not all) of rules 298. Entries associated with these parameters (5a, 6a, 8a) are still tested and scored, but are not used to determine next (L,I) values. Notice thus that entries at steps i=1-9 in FIG. 18A (in which it is assumed that the user always accepts use of the next (L,I) value) and FIG. 18C (in which the user eventually uses these next (L,I) values) are identical.

While non-rule-compliant entries in the data set 230 are not used by the algorithm 200 in helping the algorithm iterate and determine next (L,I) values to test, these entries are still relevant and assessed by the algorithm when determining Lopt and Iopt1. Thus, after reaching a stopping criterium (325), Lopt and Iopt1 are determined (630) using all scores in the data set 230, and regardless whether the user had accepted the next (L,I) value (✓); whether the user enters user-defined stimulation parameters consistent with rules 298 (−); or whether the user enters user-defined stimulation parameters inconsistent with rules 298 (X). Thus, in the example of FIG. 18C, (Lopt, Iopt1) is determined to be consistent with the stimulation parameters used at entry 6a (6.5,3.7), because these parameters produce the best (lowest) score (0.3) for the patient. Notice then that the algorithm 200 does not necessarily select (Lopt, Iopt1) using only at (L,I) values that the algorithm 200 selects or predicts.

Returning to FIG. 16, once longitudinal optimization has been completed by determining Lopt, Iopt1 (300), rotational optimization can be undertaken to determine θopt, Iopt2 at Lopt if necessary (370). As noted earlier, rotational optimization at this step (400) is modified similarly to the manner in which longitudinal optimization was modified, as shown in FIG. 19. The details of FIG. 19 should be clear and are analogous to what was described earlier with respect to FIG. 17. As noted earlier, the rules 398 used during rotational optimization may differ from the rules 298 used during longitudinal optimization. For example, rules 398 may impose an additional rule that the longitudinal position of the stimulation should be kept at or near Lopt determined earlier during longitudinal optimization.

As shown in FIG. 19 (and analogously to FIG. 17), rotational optimization at Lopt (400) will predict next (θ,I) values to test when determining θopt, Iopt2 at Lopt. If a user does not wish to accept use of these values instantly, the user can enter new user-defined stimulation parameters which are assessed for compliance with rules 398 (712). If user-defined stimulation parameters are consistent with rules 398 (712), they are applied and scored (714, 716), and used as a bases for determining next (θ,I) values to test (530). If user-defined stimulation parameters are not consistent with rules 398 (712), they are again applied and scored (722, 724), but are not used as a basis for determining next (θ,I) values to test. Stopping criteria (525) may again be skipped immediately after the algorithm 200 tests user-defined stimulation parameters.

The flow of FIG. 19 is illustrated in the example of FIG. 20, which shows how data set 230′ (compare FIG. 12) is populated during rotational optimization at step 400. Again, details are skipped, as FIG. 20 is similar to what was described earlier with respect to FIGS. 18A-18C during longitudinal optimization and should be clear based on foregoing explanations. After step i=5, it is assumed that the user doesn't accept a next (θ,I) value (240, 2.7) (530) that the algorithm 200 predicted based on earlier tested entries (708). Instead, the user enters user-defined stimulation parameters (710), which are consistent with rules 398 (712), and which correspond to (θ,I)=(300, 4.0) (at Lopt). These parameters are applied (714), scored (S′=0.2) and flagged (−) (716). Further, because these user-defined stimulation parameters are consistent with rules 398, this score (step i=6) is used when determining a next (θ,I) value to test (60, 0.5) (530). The user accepts, applied, and scores this next (θ,I) value at step i=7, and another next (θ,I) value is determined (150, 3.7) (530).

At this point, the user does not accept the next (θ,I) value (150,3.7) (708), and again enters user-defined stimulation parameters (710). This time, the user-defined stimulation parameters are not consistent with rules 398 (712), in particular because stimulation provided by these parameters is not applied at longitudinal position Lopt (1.5) (or within a permissible range around Lopt). Instead, they define stimulation at a different longitudinal position (L=5). The user-defined stimulation parameters would also not be consistent with rules 398 if the frequency, pulse width, polarity were changed, or if corresponding values for θ or I are excluded from the θ,I parameter space 230′ being tested, but this isn't illustrated in FIG. 20. In any event, the user may be notified of the inconsistency (720), and the user-defined stimulation parameters applied (722), scored (S′=1.6), and flagged (724) at entry 7a. This score would not be used in determining a next (or any future) (θ,I) value to test (at 530), so the score at this point remains (150, 3.7). This score is eventually accepted, tested, and scored (step i=8).

Step i=9 shows another example in which the user does not accept a next (θ,I) value (708), and enters user-defined stimulation parameters (710) which again specify stimulation at a longitudinal position (L=1.7) different from Lopt=1.5. This time however, the user-defined stimulation parameters are considered to be consistent with rules 398 (712), because L is reasonably close to Lopt, e.g., within a range specified by rules 398. As such, these parameters will be applied (714), scored, and flagged (−) (716). Thus, this score (S′=1.9) will be used by the algorithm 200 in determining a next (θ,I) value to test (at step i=10). This may be because (as specified in rules 398) the scores S′ being used by the algorithm reflect side effects at least in part, as opposed to raw efficacy. In this circumstance, rules 398 may consider it reasonable for the user to modify L slightly (e.g., Lopt−Δ<L<Lopt+Δ), and will consider scores procured from testing at those values to be relevant enough to use when predicting next (θ,I) values. The algorithm 200 could however treat step i=9 differently. For example, if the scores S′ used by the algorithm 200 only reflect efficacy, rules 398 may prescribe that no variation in L from Lopt is permissible. In this circumstance, if the user enters a L not equal to Lopt, the algorithm 200 would treat these user-defined stimulation parameters as inconsistent with rules (712), and would apply (722), and score them (724), but such scores would be flagged differently (X) and not used as bases for predicting next (θ,I) values.

The algorithm 200 determines θopt, Iopt2, Lopt based on the best (lowest) recorded score in data set 230′ (730), which in this example is at step i=6 (θopt=300, Iopt2=4.0, and Lopt=1.5), which provides the best (lowest) score (S′=0.2), and hence determines the optimal stimulation parameters for the patient. Notice that Lopt may vary from Lopt determined earlier (1.5), which could happen if the user entered user-defined stimulation parameters (710) that were not at or near the value of Lopt determined earlier (and hence not within rules 398; e.g., entry 7a), and if these parameters happen to have the best score.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system, comprising:
a non-transitory computer-readable medium comprising an algorithm configured to be executed by control circuitry of an external system configured to program an implantable stimulator device (ISD) of a patient, wherein the ISD comprises a lead with a plurality of electrodes for providing stimulation in accordance with a plurality of stimulation parameters, wherein the algorithm is configured to:
allow a user to define or accept one or more rules for the stimulation parameters, wherein the one or more rules are consistent with a parameter space to be tested;
iteratively test sets of stimulation parameters, wherein at each iteration the algorithm is configured to
present a set of stimulation parameters to a user within the parameter space,
receive either (i) an indication that the user accepts the set of stimulation parameters, or (ii) user-defined stimulation parameters,
assess compliance of the user-defined stimulation parameters, if received, with the one or more rules,
program the ISD to provide stimulation using (i) the set of stimulation parameters if accepted, else (ii) the user-defined stimulation parameters, and
record an entry comprising the stimulation and at least one score indicative of the efficacy of the simulation,
flag the entry with a first flag if the stimulation was provided using the user-defined stimulation parameters and if the user-defined stimulation parameters are not compliant with the one or more rules, and
determine a next set of stimulation parameters to test within the parameter space using entries that are not flagged with the first flag; and
determine as optimal stimulation for the patient the stimulation associated with the entry having a best score.

2. The system of claim 1, wherein the score is indicative of the efficacy of the stimulation in treating a symptom of the patient and/or a side effect caused by the stimulation.

3. The system of claim 1, wherein at least one of the one or more rules comprises a rule requiring the stimulation to have a pulse width or frequency that is constant.

4. The system of claim 1, wherein at least one of the one or more rules comprises a rule requiring the stimulation to have a consistent polarity.

5. The system of claim 1, wherein the parameter space comprises a range of longitudinal positions on the lead.

6. The system of claim 5, wherein at least one of the one or more rules comprises a rule requiring the stimulation to have a longitudinal position within the range of longitudinal positions.

7. The system of claim 5, wherein at least one of the one or more rules comprises a rule requiring the stimulation to be symmetric around the lead.

8. The system of claim 5, wherein the parameter space further comprises a range of amplitudes.

9. The system of claim 8, wherein at least one of the one or more rules comprises a rule requiring the stimulation to have an amplitude with the range of amplitudes.

10. The system of claim 1, wherein determining the next set of stimulation parameters to test comprises determining a longitudinal position along the lead and an amplitude.

11. The system of claim 1, wherein the parameter space comprises a range of rotational positions around the lead.

12. The system of claim 11, wherein at least one of the one or more rules comprises a rule requiring the stimulation to have a rotational position within the range of rotational positions.

13. The system of claim 11, wherein the parameter space further comprises a range of amplitudes, wherein at least one of the one or more rules comprises a rule requiring the stimulation to have an amplitude with the range of amplitudes.

14. The system of claim 11, wherein at least one of the one or more rules comprises a rule requiring the stimulation to be at a longitudinal position along the lead, or within a range of longitudinal position along the lead.

15. The system of claim 1, wherein determining the next set of stimulation parameters to test comprises determining a rotational position around the lead and an amplitude.

16. The system of claim 1, wherein, during iteratively testing the sets of stimulation parameters, the algorithm is further configured to selectively determine if one or more stopping criterium have been met, and thus stop iteratively testing the sets of stimulation parameters, before determining the optimal stimulation for the patient.

17. The system of claim 16, wherein if the stimulation was provided using the user-defined stimulation parameters, the algorithm is configured to not determine if the one or more stopping criterium have been met.

18. The system of claim 1, further comprising the ISD.

19. The system of claim 1, further comprising the external system.

20. A method performed on an external system configured to program an implantable stimulator device (ISD) of a patient, wherein the ISD comprises a lead with a plurality of electrodes for providing stimulation in accordance with a plurality of stimulation parameters, the method comprising:
allowing a user to define or accept at the external system one or more rules for the stimulation parameters, wherein the one or more rules are consistent with a parameter space to be tested;
iteratively testing sets of stimulation parameters, wherein at each iteration the method
presents at the external system a set of stimulation parameters to a user within the parameter space;
receives either (i) an indication that the user accepts the set of stimulation parameters, or (ii) user-defined stimulation parameters;
assesses compliance of the user-defined stimulation parameters, if received, with the one or more rules;
programs the ISD to provide stimulation using (i) the set of stimulation parameters if accepted, else (ii) the user-defined stimulation parameters, and
records an entry comprising the stimulation and at least one score indicative of the efficacy of the simulation,
flags the entry with a first flag if the stimulation was provided using the user-defined stimulation parameters and if the user-defined stimulation parameters are not compliant with the one or more rules; and
determines a next set of stimulation parameters to test within the parameter space using entries that are not flagged with the first flag; and
determining, at the external system, as optimal stimulation for the patient the stimulation associated with the entry having a best score.
